(19) United States
(12) Patent Application Publication
(10) Pub. No.: US 2022/0008537 A1
(43) Pub. Date: Jan. 13, 2022

(54) COMPOUNDS FOR PHOTODYNAMIC THERAPY AND RELATED USES

(71) Applicant: GEORGIA STATE UNIVERSITY RESEARCH FOUNDATION, INC., Atlanta, GA (US)

(72) Inventors: Maged HENARY, Lawrenceville, GA (US); Kathryn GRANT, Atlanta, GA (US)

(21) Appl. No.: 17/295,162

(22) PCT Filed: Nov. 19, 2019
(86) PCT No.: PCT/US2019/062210

§ 371 Date: May 19, 2021

Related U.S. Application Data
(60) Provisional application No. 62/769,301, filed on Nov. 19, 2018.

(51) Int. Cl. A61K 41/00; A61P 35/00

(52) U.S. Cl. CPC A61K 41/0057; A61P 35/00

(57) ABSTRACT

Disclosed herein are symmetrical and unsymmetrical carbocyanine dyes. Irradiation of the dyes generates reactive species which contribute to DNA damage. The dyes are useful for the treatment of various cancers and cell growth disorders.

### CROSS-REFERENCE TO RELATED APPLICATION

This application claims the benefit of U.S. Provisional Application 62/769,301, filed Nov. 19, 2018, the contents of which are hereby incorporated in its entirety.

### FIELD OF THE INVENTION

The invention is directed to photosensitizing compounds useful for photodynamic therapy.

### BACKGROUND

Photodynamic therapy (PDT) is an approach to cancer treatment aimed at minimizing the severe side effects associated with traditional chemotherapeutic agents. In PDT, excitation of a photosensitizer (PS) with low energy light triggers the production of highly localized reactive oxygen species (ROS) in diseased tissues with minimal involvement of surrounding cells. In the most common PDT mechanism, singlet oxygen (¹O₂) is generated by Type 2 energy transfer between the triplet excited state (³PS*) of the PS and ground state triplet oxygen (³O₂). The triplet state can also react with ³O₂ by Type I electron transfer to yield superoxide anion radicals (O₂.⁻). Spontaneous dismutation of O₂.⁻ generates H₂O₂, which gives rise to hydroxyl radicals (.OH) by a Fenton reaction. With respective diffusion distances of 50-100 nm and 0.8-6.0 nm, the short-lived and highly reactive ¹O₂ and .OH formed upon dye excitation cause extensive oxidative damage to DNA and other cellular macromolecules in their vicinity.

The commonly used PDT agents porfimer sodium, talaporfin, and verteporfin directly sensitize cleavage of genomic DNA when irradiated in tissue culture and/or in circulating cells. While their absorption bands are compatible with visible light sources that emit at wavelengths ≤689 nm, alternative photosensitizers that possess near-infrared maxima extending from ˜700 nm to 900 nm are desired. This is due to enhanced penetration of incident irradiation afforded by minimal absorption of light in this range by molecules in the body. The light depth attained at 835 nm through biological tissue is approximately twice that at 630 nm, the wavelength used to activate porfimer sodium.

Although infrared light penetrates tissues deeply, red-shifting the λₘₐₓ of a chromophore reduces triplet state energy, placing limits on near-IR ROS production. Type 2 singlet oxygen is generated only when a PS has a triplet state energy equivalent to or higher than the excitation energy of ¹O₂ (95 kJmol-1, ˜1270 nm). When taking into consideration the minimal energy gap between the first excited ¹PS* and ³PS* states of PDT agents (≤63 kJmol-1), this translates into a ˜810 nm upper absorption limit for ¹O₂ production. In order for a photosensitizer to form Type 1 superoxide, the oxidation potential of its triplet state should be higher than the oxidation potential of ground state triplet oxygen (E° (³O₂/O₂.⁻)=0.16 V at pH 7.0), but excited state oxidation potentials decrease as a function of decreasing triplet state energy. As a result, there are relatively few examples of DNA photocleaving agents that are effective ROS generators in the near-infrared range. Using an anthracenyl-bis(pyridyl)Fe(III) catecholate complex to sensitize hydroxyl radical production, Chakravarty and co-workers cleaved plasmid DNA in high yield under 785 nm illumination. Until the present report, this was the longest wavelength ever to have been used to trigger DNA cleavage upon direct, single photon chromophore sensitization.

Near-infrared cyanine dyes are currently being developed as PDT agents and are used in clinical settings as fluorescent probes in the diagnosis and imaging of cancer. DNA interactions are facilitated by the cyanines' two flanking heteroaromatic nitrogen rings, which share a positive charge that is delocalized through a central polymethine bridge. DNA cleavage by hydroxyl radicals and singlet oxygen has been reported, with excitation wavelengths in the visible range up to 700 nm. A number of cyanine dyes, particularly those with 2-quinoline ring systems, avidly interact with the DNA minor groove as monomers, dimers, and higher order aggregates.

There remains a need for improved methods of photodynamic therapy. There remains a need for improved compounds for use in photodynamic therapy. There remains a need for photodynamic compounds capable of undergoing single photon excitation at longer wavelengths.

### SUMMARY

Disclosed herein are compounds for use in photodynamic therapy. The compounds are defined by the chemical formula:

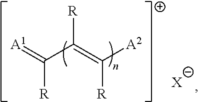

wherein A¹, A², R, and X are defined herein.

The details of one or more embodiments are set forth in the descriptions below. Other features, objects, and advantages will be apparent from the description and from the claims.

### BRIEF DESCRIPTION OF THE FIGURES

FIGS. 1A-1B: UV-visible spectra of 10 μM of dyes 4 (FIG. 1A) and 5(FIG. 1A) in DMSO (t=0 mM); 10 mM sodium phosphate pH 7.0 buffer, without DNA (t=0 min) and with 150 μM bp CT DNA (t=0 to t=25 min).

FIGS. 2A-2B: Agarose gels showing cyanine dye-sensitized photocleavage of pUC19 plasmid DNA irradiated with 808 nm (FIG. 2A) and 830 nm (FIG. 2B) LED lamps (2.8 W/cm², 30 min hv at 10° C.). Reactions contained 10 mM sodium phosphate buffer pH 7.0 and 38 μM bp DNA in the absence and presence of 20 μM of dye. Yields and standard deviation were obtained over 3 trials. Abbreviations: L=linear; N=nicked; S=supercoiled.

FIG. 3: Double y-axis plots superimposing the circular dichroism (CD) and UV-visible absorption (Abs) spectra of dye 5 (22° C.). Samples contained 10 mM sodium phosphate buffer pH 7.0, 10 μM of dye and/or 120 μM bp (CD) to 150 μM bp (Abs) of CT DNA.

FIGS. 4A-4B: Fluorescence spectra recorded in 10 mM sodium phosphate buffer pH 7.0 without and with 20 μM of 5 and: (FIG. 4A) 3 μM HPF (ex=490 nm)±100 mM sodium benzoate (SB); (FIG. 4B) 0.75 μM SOSG (λex=480 nm). Reactions were kept in the dark or irradiated (hv) at 830 nm for 30 min (22° C.).

FIGS. 5A-5B: (FIG. 5A) Representative superimposed fluorescence microscopy images reveal intracellular localization of dye 5 in ES2 cancer cells after incubation for 24 h followed by staining nuclei with Hoechst 33342. (FIG. 5B) ES2 cancer cell viability for: Cells—no treatment; Light-cells exposed to a 808 nm laser (0.3 W/cm2) for 10 min; (5)−cells incubated with dye 5 (10 μg/mL=20 μM) for 24 h under dark conditions; (5)+Light—cells incubated with dye 5 (10 μg/mL) for 24 h and exposed to a 808 nm laser (0.3 W/cm²) for 10 min. *p<0.05 when compared with non-treated cells.

FIGS. 6A-6B: UV-visible spectra recorded as a function of time for 10 μM of dyes 4 (FIG. 6A) and 5 (FIG. 6B) in DMSO (22° C.).

FIGS. 7A-7D: UV-visible spectra recorded as a function of time for 10 μM of dyes 4 (FIGS. 7A and 7C) and 5 (FIGS. 7B and 7D) in the absence and presence of 150 μM bp CT DNA (10 mM sodium phosphate buffer pH 7.0; 22° C.).

FIGS. 8A-8B: Agarose gels showing cyanine dye-sensitized photocleavage of pUC19 plasmid DNA with (FIG. 8A) 808 nm and (FIG. 8B) 830 nm LED lamps (2.8 W/cm²; 30 min hv at 22° C.). Reactions contained 10 mM sodium phosphate buffer pH 7.0 and 38 μM bp DNA in the absence and presence of 20 μM of dye. Yields and standard deviation were obtained over 3 trials. Abbreviations: L=linear; N=nicked; S=supercoiled).

FIGS. 9A-9C: Agarose gels showing controls in which cyanine dyes 4 and 5 were equilibrated with pUC19 plasmid DNA in the dark at temperatures at 10° C. (FIG. 9A), 10° C. (FIG. 9B), and 37° C. (FIG. 9C) (30 mM no hv). The reactions contained 10 mM sodium phosphate buffer pH 7.0 and 38 μM bp DNA in the absence and presence of 20 μM of dye. Abbreviations: L=linear; N=nicked; S=supercoiled).

FIGS. 10A-10B: Agarose gel (FIG. 10A) and corresponding yields (FIG. 10B) showing photocleavage of 38 μM bp pUC19 DNA by 0 to 50 μM of dye 5 (10 mM sodium phosphate buffer pH 7.0). With the exception of the dark controls in lane 1, reactions were irradiated with a 830 nm LED lamp (2.8 W/cm²) for 30 mM at 22° C. Abbreviations: N=nicked; S=supercoiled.

FIG. 11: Photocleavage of 38 μM bp pUC19 DNA by 20 μM of dye 5 (10 mM sodium phosphate buffer pH 7.0). Reactions were irradiated with a 830 nm LED lamp for 0, 1, 5, 10, 15, 20, 25, 30, 60, 90, and 120 mM time intervals at 10° C. (2.8 W/cm²). Data points are averaged over three trials. Error bars represent standard deviation. Abbreviations: N=nicked; S=supercoiled.

FIG. 12: Representative UV-visible absorption titration spectra of 20 μM of cyanine dye 5 in the absence and presence of increasing concentrations of CT DNA (10 mM sodium phosphate buffer, pH 7.0, 22° C.). All absorption spectra were corrected for sample dilution.

FIG. 13: Double y-axis plots superimposing the extended, near-infrared circular dichroism (CD) and UV-visible absorption (Abs) spectra of dye 5 (22° C.). Samples contained 10 mM sodium phosphate buffer pH 7.0, 20 μM (Abs) or 25 μM (CD) of dye and 990 μM bp of CT DNA.

FIGS. 14A-14B: Double y-axis plots superimposing the fluorescence emission (Em) and UV-visible absorption (Abs) spectra of dye 5 (22° C.). Samples contained 10 mM sodium phosphate buffer pH 7.0, 10 μM (Em) or 20 μM (Abs) of dye and/or 100 μM to 990 μM bp of CT DNA. The emission spectra were recorded at excitation wavelengths (Ex) of 550 nm (FIG. 14A) and 800 nm (FIG. 14B).

FIGS. 15A-15B: Agarose gels showing cyanine dye-sensitized photocleavage of pUC19 plasmid DNA (FIG. 15A) after 30 mM of irradiation with a 532 nm LED laser (1.0 W/cm²) and (FIG. 15B) after a 30 min incubation period in the dark (10° C.). Reactions contained 10 mM sodium phosphate buffer pH 7.0 and 38 μM bp DNA in the absence and presence of 20 μM of dye 5. Abbreviations: N=nicked; S=supercoiled).

FIG. 16: Agarose gel showing cyanine dye-sensitized photocleavage of pUC19 plasmid DNA under aerobic and anaerobic conditions. Reactions containing 10 mM sodium phosphate buffer pH 7.0, 20 μM of dye 5, and 38 μM bp DNA were purged in a glove box with air or argon and then either irradiated with a 830 nm LED lamp (2.8 W/cm²) or kept in the dark in the purged glove box (30 min, 22° C.). Abbreviations: L=linear; N=nicked; S=supercoiled.

FIGS. 17A-17C: Agarose gels comparing levels of cyanine dye-sensitized photocleavage of pUC19 plasmid DNA generated in the absence (FIG. 17A) and presence of the ROS scavenging agents sodium benzoate (FIG. 17B), and sodium azide (FIG. 17C) (830 nm hv for 30 min at 22° C.). All reactions contained 10 mM sodium phosphate buffer pH 7.0, 20 μM of dye 5, and 38 μM bp DNA. Abbreviations: L=linear; N=nicked; S=supercoiled.

FIGS. 18A-18B: Agarose gels showing cyanine dye-sensitized photocleavage of pUC19 plasmid DNA in 100% H₂O (v/v) (FIG. 18A) vs. 70% D₂O (v/v) (FIG. 18B). The reactions, which contained 10 mM sodium phosphate buffer pH 7.0 and 38 μM bp DNA in the absence and presence of 20 μM of dye 5, were either kept in the dark or irradiated with a 830 nm LED lamp (2.8 W/cm2, 30 min hv at 22° C.). Abbreviations: L=linear; N=nicked; S=supercoiled.

FIGS. 19A-19B: Fluorescence spectra recorded at 22° C. of: (FIG. 19A) 3 μM hydroxyphenyl fluorescein (HPF) in the absence and presence of either 10 μM ammonium iron(II) sulphate/10 μM H₂O₂ or 10 μM ammonium iron(II) sulphate/10 μM H₂O₂ and 100 mM sodium benzoate (SB); (FIG. 19B) 0.75 μM Singlet Oxygen Sensor Green® (SOSG) in the absence and presence of either 10 μM ammonium iron(II) sulphate/10 μM H₂O₂, 1 μM methylene blue, or 1 μM methylene blue irradiated for 2 s with a 638 nm LED laser (2.8 W/cm², Laserland). All samples contained 10 mM sodium phosphate buffer pH 7.0.

FIG. 20: Relative intracellular ROS levels detected by DCFH-DA in ES2 cancer cells after the following treatments: Cells—no treatment; Light-cells exposed to a 808 nm laser (0.3 W/cm²) for 5 min; (5)−cells incubated with dye 5 (1 μg/mL) for 24 h under dark conditions; (5)+Light—cells incubated with dye 5 (1 μg/mL) for 24 h and exposed to a 808 nm laser (0.3 W/cm2) for 5 min. ROS level of non-treated cells was set to 1. *p<0.05 when compared with non-treated cells.

FIG. 21: In vitro dark toxicity curves of various dyes. ES2 cells ovarian cancer cells were grown overnight and then incubated with the cyanine dyes for 24 hours. A calcein-based fluorometric assay was then used to quantitate percent cell survival. The results show that the cyanine dyes are non-toxic in the dark at dye concentrations ranging from 0.0005 microgram per mL to 1 microgram per mL.

FIGS. 22A-22B: Activity of certain dyes to ES2 cells. FIG. 22A) In vitro photo-toxicity curves of various dyes. ES2 cells ovarian cancer cells were plated at 10 k cells/well and incubated overnight. Solubilized dye was then put into the wells at a final concentration of 0.5 microgram per mL and left for 24 hours. The wells were exposed to lasers as follows (5 min/well): IV-A 780 nm laser, ˜0.6 W/cm²; VI-A 780 nm laser, ˜0.6 W/cm²; III-A 830 nm laser, ˜0.6 W/cm²; II-A 830 nm laser, ˜0.6 W/cm²; V-A 694 nm laser, ˜1.3 W/cm²; VII-A 694 nm laser, ˜1.3 W/cm². A calcein-based fluorometric assay was then used to quantitate percent cell survival. The results show that the cyanine dyes become photo-toxic to ES2 ovarian cancer cells when irradiated in the near-infrared wavelength range. FIG. 22B) ES2 cells were plated at 10 k cells/well and left overnight. The solubilized dyes were then put into the wells, left for 24 hours, and lasered as described above. A H2DCFDA-based fluorometric assay was then used to quantitate the intracellular reactive oxygen species generated by the irradiated cyanine dyes.

FIG. 23: Agarose gels showing cyanine dye-sensitized photocleavage of pUC19 plasmid DNA irradiated with 830 nm, 850 nm, and 905 nm LED lasers (2.8 W/cm², 30 min hv at 10° C.). Reactions contained 10 mM sodium phosphate buffer pH 7.0 and 38 μM bp DNA in the absence and presence of 50 μM of cyanine dye I-A or I-E. Abbreviations: L=linear; N=nicked; S=supercoiled; B=plasmid DNA in the dark; D=plasmid DNA+compound in the dark. The results show that cyanine dye I-A generates strong DNA photo-cleavage under all near-infrared light wavelengths tested with low levels of DNA damage in dark control reactions.

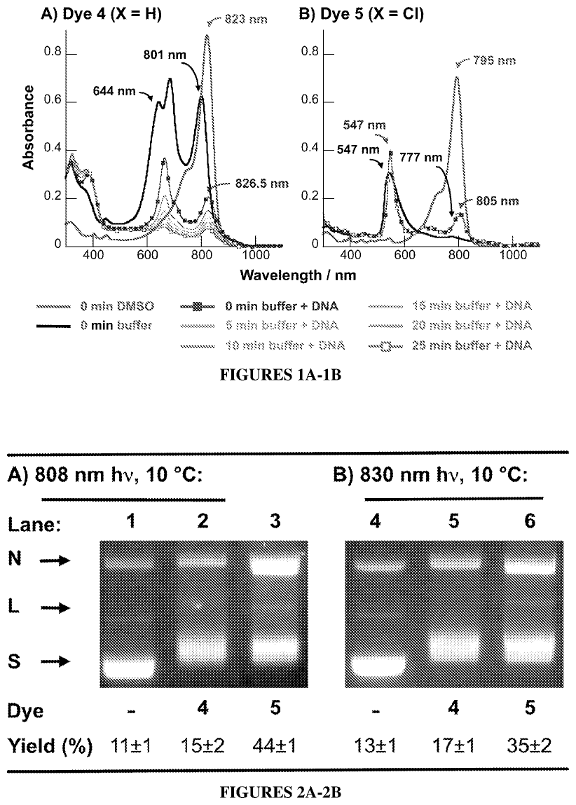

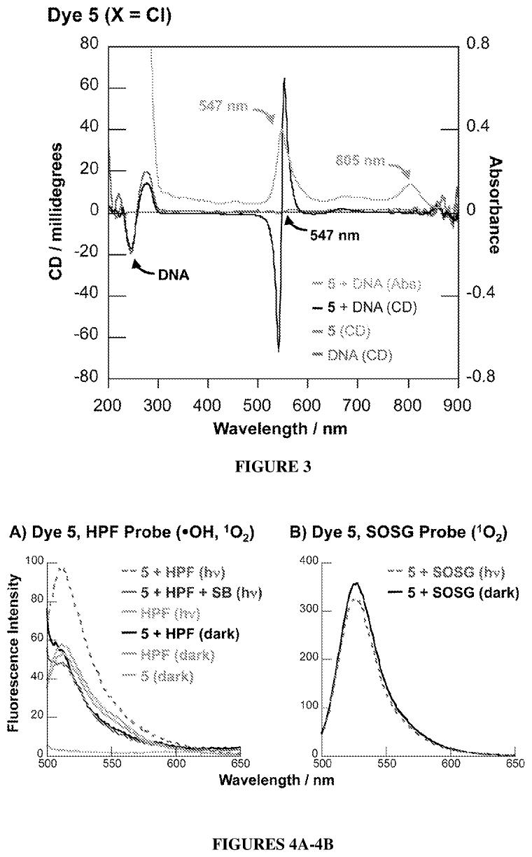

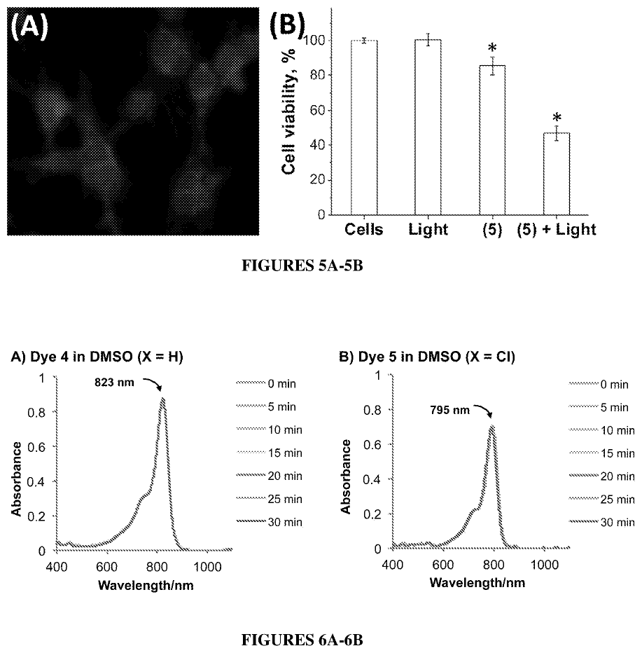

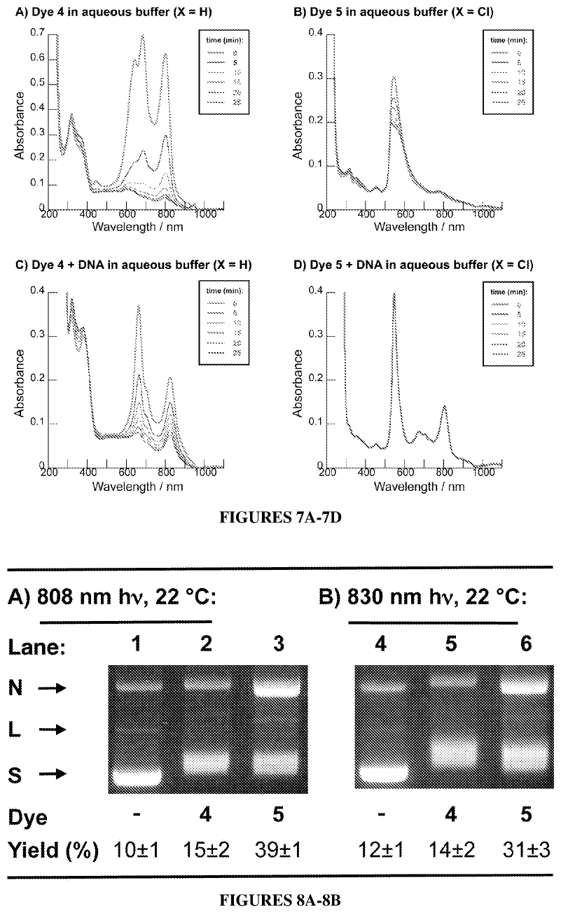

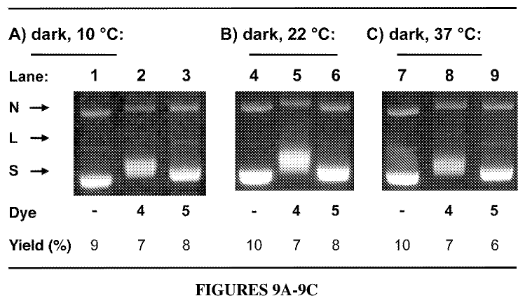

### DETAILED DESCRIPTION

Before the present methods and systems are disclosed and described, it is to be understood that the methods and systems are not limited to specific synthetic methods, specific components, or to particular compositions. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another embodiment includes¬ from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal embodiment. “Such as” is not used in a restrictive sense, but for explanatory purposes.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

The term “alkyl” as used herein is a branched or unbranched hydrocarbon group such as methyl, ethyl, n-propyl, isopropyl, n-butyl, isobutyl, t-butyl, pentyl, hexyl, heptyl, octyl, nonyl, decyl, dodecyl, and the like. The alkyl group can also be substituted or unsubstituted. Unless stated otherwise, the term “alkyl” contemplates both substituted and unsubstituted alkyl groups. The alkyl group can be substituted with one or more groups including, but not limited to, alkoxy, alkenyl, alkynyl, cycloalkyl, heterocycloalkyl, aryl, heteroaryl, aldehyde, amino, carboxylic acid, ester, ether, halide, hydroxy, ketone, nitro, silyl, sulfo-oxo, phosphine or thiol. An alkyl group which contains no double or triple carbon-carbon bonds is designated a saturated alkyl group, whereas an alkyl group having one or more such bonds is designated an unsaturated alkyl group. Unsaturated alkyl groups having a double bond can be designated alkenyl groups, and unsaturated alkyl groups having a triple bond can be designated alkynyl groups. Unless specified to the contrary, the term alkyl embraces both saturated and unsaturated groups.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, etc. The term “heterocycloalkyl” is a cycloalkyl group as defined above where at least one of the carbon atoms of the ring is replaced with a heteroatom such as, but not limited to, nitrogen, oxygen, sulfur, selenium or phosphorus. The cycloalkyl group and heterocycloalkyl group can be substituted or unsubstituted. Unless stated otherwise, the terms “cycloalkyl” and “heterocycloalkyl” contemplate both substituted and unsubstituted cyloalkyl and heterocycloalkyl groups. The cycloalkyl group and heterocycloalkyl group can be substituted with one or more groups including, but not limited to, alkyl, alkoxy, alkenyl, alkynyl, cycloalkyl, heterocycloalkyl, aryl, heteroaryl, aldehyde, amino, carboxylic acid, ester, ether, halide, hydroxy, ketone, nitro, silyl, sulfo-oxo, or thiol. A cycloalkyl group which contains no double or triple carbon-carbon bonds is designated a saturated cycloalkyl group, whereas an cycloalkyl group having one or more such bonds (yet is still not aromatic) is designated an unsaturated cycloalkyl group. Unless specified to the contrary, the term cycloalkyl embraces both saturated and unsaturated, non-aromatic, ring systems.

The term “aryl” as used herein is an aromatic ring composed of carbon atoms. Examples of aryl groups include, but are not limited to, phenyl and naphthyl, etc. The term “heteroaryl” is an aryl group as defined above where at least one of the carbon atoms of the ring is replaced with a heteroatom such as, but not limited to, nitrogen, oxygen, sulfur, selenium or phosphorus. The aryl group and heteroaryl group can be substituted or unsubstituted. Unless stated otherwise, the terms “aryl” and “heteroaryl” contemplate both substituted and unsubstituted aryl and heteroaryl groups. The aryl group and heteroaryl group can be substituted with one or more groups including, but not limited to, alkyl, alkoxy, alkenyl, alkynyl, cycloalkyl, heterocycloalkyl, aryl, heteroaryl, aldehyde, amino, carboxylic acid, ester, ether, halide, hydroxy, ketone, nitro, silyl, sulfo-oxo, or thiol.

Exemplary heteroaryl and heterocyclyl rings include: benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH carbazolyl, carbolinyl, chromanyl, chromenyL cirrnolinyl, decahydroquinolinyl, 2H,6H˜1,5,2-dithiazinyl, dihydrofuro[2,3 b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, and xanthenyl.

The terms “alkoxy” and “alkoxyl” as used herein to refer to an alkyl or cycloalkyl group bonded through an ether linkage; that is, an “alkoxy” group can be defined as—OA¹ where A¹ is alkyl as defined above. “Alkoxy” also includes polymers of alkoxy groups as just described; that is, an alkoxy can be a polyether such as —OA¹—OA² or —OA¹-(OA²)ₐ-OA³, where “a” is an integer of from 1 to 200 and A¹, A², and A³ are alkyl groups.

The terms “cycloalkoxy,” “heterocycloalkoxy,” “cycloalkoxy,” “aryloxy,” and “heteroaryloxy” have the aforementioned meanings for alkyl, cycloalkyl, heterocycloalkyl, aryl and heteroaryl, further providing said group is connected via an oxygen atom.

As used herein, the term “null,” when referring to a possible identity of a chemical moiety, indicates that the group is absent, and the two adjacent groups are directly bonded to one another. By way of example, for a genus of compounds having the formula CH₃—X—CH₃, if X is null, then the resulting compound has the formula CH₃—CH₃.

As used herein, two atoms connected via the symbol [symbol] may be connected via a single or double bond.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valencies of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. Unless specifically stated, a substituent that is said to be “substituted” is meant that the substituent can be substituted with one or more of the following: alkyl, alkoxy, alkenyl, alkynyl, cycloalkyl, heterocycloalkyl, aryl, heteroaryl, aldehyde, amino, carboxylic acid, ester, ether, halide, hydroxy, ketone, nitro, silyl, sulfo-oxo, phosphine, or thiol. In a specific example, groups that are said to be substituted are substituted with a protic group, which is a group that can be protonated or deprotonated, depending on the pH.

Unless specified otherwise, the term “patient” refers to any mammalian animal, including but not limited to, humans.

Pharmaceutically acceptable salts are salts that retain the desired biological activity of the parent compound and do not impart undesirable toxicological effects. Examples of such salts are acid addition salts formed with inorganic acids, for example, hydrochloric, hydrobromic, sulfuric, phosphoric, and nitric acids and the like; salts formed with organic acids such as acetic, oxalic, tartaric, succinic, maleic, fumaric, gluconic, citric, malic, methanesulfonic, p-toluenesulfonic, napthalenesulfonic, and polygalacturonic acids, and the like; salts formed from elemental anions such as chloride, bromide, and iodide; salts formed from metal hydroxides, for example, sodium hydroxide, potassium hydroxide, calcium hydroxide, lithium hydroxide, and magnesium hydroxide; salts formed from metal carbonates, for example, sodium carbonate, potassium carbonate, calcium carbonate, and magnesium carbonate; salts formed from metal bicarbonates, for example, sodium bicarbonate and potassium bicarbonate; salts formed from metal sulfates, for example, sodium sulfate and potassium sulfate; and salts formed from metal nitrates, for example, sodium nitrate and potassium nitrate. Pharmaceutically acceptable and non-pharmaceutically acceptable salts may be prepared using procedures well known in the art, for example, by reacting a sufficiently basic compound such as an amine with a suitable acid comprising a physiologically acceptable anion. Alkali metal (for example, sodium, potassium, or lithium) or alkaline earth metal (for example, calcium) salts of carboxylic acids can also be made.

The compounds disclosed herein are broadly useful as photosensitizers, for instance as photosensitizers for use in photodynamic therapy. In some embodiments, the compounds can have an absorption maxima of at least 700 nm, at least 725 nm, at least 750 nm, at least 775 nm, at least 800 nm, at least 810 nm, at least 820 nm, at least 830 nm, at least 840 nm, at least 850 nm, at least 860 nm, at least 870 nm, at least 880 nm, at least 890 nm, at least 1,000 nm, at least 1,010 nm, at least 1,020 nm, at least 1,030 nm, at least 1,040 nm, at least 1,050 nm, at least 1,060 nm, at least 1,070 nm, at least 1,080 nm, at least 1,090 nm, or at least 1,100 nm. In some embodiments, the compounds disclosed herein can have an absorption maxima between 600-900 nm, between 650-900 nm, between 700-900 nm, between 725-900 nm, between 750-900 nm, between 775-900 nm, between 800-900 nm, between 825-900 nm, between 825-875 nm, between 850-900 nm, between 700-850 nm, between 750-850 nm, or between 800-850 nm. In other instances, the compounds disclosed herein can have an absorption maxima between 1,000-1,200 nm, between 1,000-1,150 nm, between 1,000-1,100 nm, between 1,000-1,050, between 1,025-1,075 nm, or between 1,050-1,100.

In some embodiments, the compounds disclosed herein do not strongly fluoresce when irradiated at the wavelengths described above. For instance, the compounds disclosed herein can have a fluorescence, as measured by quantum yield relative to quinine sulfate in sulfuric acid solution, no greater than 0.02 Φ, no greater than 0.01 Φ, no greater than 0.005 Φ, no greater than 0.001 Φ, no greater than 0.0005 Φ, no greater than 0.0001 Φ, no greater than 0.00005 Φ, or no greater than 0.00001 Φ.

In some embodiments, the compounds for use in photodynamic therapy can have the formula:

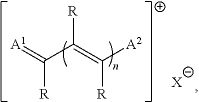

wherein A¹ and A² are individually selected from a heteroaryl ring system. Exemplary heteroaryl ring systems include pyridine, and benzofused analogs thereof, e.g., quinolines, isoquinolines, 1H-perimidines, acridines, phenantridines, and the like. In other embodiments, the heteroaryl ring system can include pyrrole, benzofused pyrroles, furans, benzofused furans, thiophenes, and benzothiophenes. In yet further embodiments, the heteroaryl ring system can include more than one heteroatoms. Such systems include phthalazines, cinnolines, napththryridines, purines, pteridines, quinazolines, quinoxalines, pyrimidines, indazoles, and the like;

R is in each case independently selected from F, Cl, Br, I, NO₂, CN, R^{a}, OR^{a}, N(R^{a})₂, SO₂R^{a}, SO₂N(R^{a})₂, C(O)R^{a}; C(O)OR^{a}, OC(O)R^{a}; C(O)N(R^{a})₂, N(R^{a})C(O)R^{a}, OC(O)N(R^{a})₂, N(R^{a})C(O)N(R^{a})₂, wherein R^{a} is in each case independently selected from hydrogen, C₁₋₈alkyl, C₂₋₈alkenyl, C₂₋₈alkynyl, aryl, C₁₋₈heteroaryl, C₃₋₈cycloalkyl, or C₁₋₈heterocyclyl. When R is a substituted C₁₋₈alkyl, C₂₋₈alkenyl, C₂₋₈alkynyl, aryl, C₁₋₈heteroaryl, C₃₋₈cycloalkyl, or C₁₋₈heterocyclyl group, said R groups may be substituted by one or more mitochondria targeting groups, e.g., phosphines, dequaliniums, rhodamines and mitochondrial penetrating peptides.

X is a pharmaceutically acceptable anion.

n can be any interger, for instance, n can be an integer from 1-5. In certain preferred embodiments, n is 1, 2 or 3.

In some embodiments, the compounds can include one or more chelating ligands. As used herein, a chelating ligand is a moiety having at least 2 chelating heteroatoms (e.g., oxygen, nitrogen, sulfur), disposed from one another by 2 or 3 atoms. Such systems bind metals such as copper and iron by forming five and six member rings that include the chelating heteroatoms and the metal. Exemplary systems include 1,2 diols, 1,3 diols, ethylene glycol ethers, propylene glycol ethers, 1,2 dicarbonyls, 1,3 dicarbonyl, 1,2 ethylene diamines, 1,3 propylene diamines, a-hydroxy carbonyls, β-hydroxy carbonyls, α-amino carbonyls, and β-amino carbonyls. In some instances, the chelating ligand can be a crown ether, thia-crown ether, or aza-crown ether, for instance [9]-crown-3; [12]-crown-4, [15]-crown-5; and [18]-crown-6. In this nomenclature, [X] refers to the total number of atoms in the ring system, and the subsequent number refers to the total number of heteroatoms. In some instances, the crown ether will contain only a single type of heteroatom, while in other cases a mixed crown ethers, e.g., those including both oxygen and nitrogen in the ring system, can be employed.

In some embodiments, A¹ can be a heterocycle having the formula represented by A^{1a}, A^{1b}, A^{1c}, A^{1d}, A^{1e}, A^{1f} or A^{1g}:

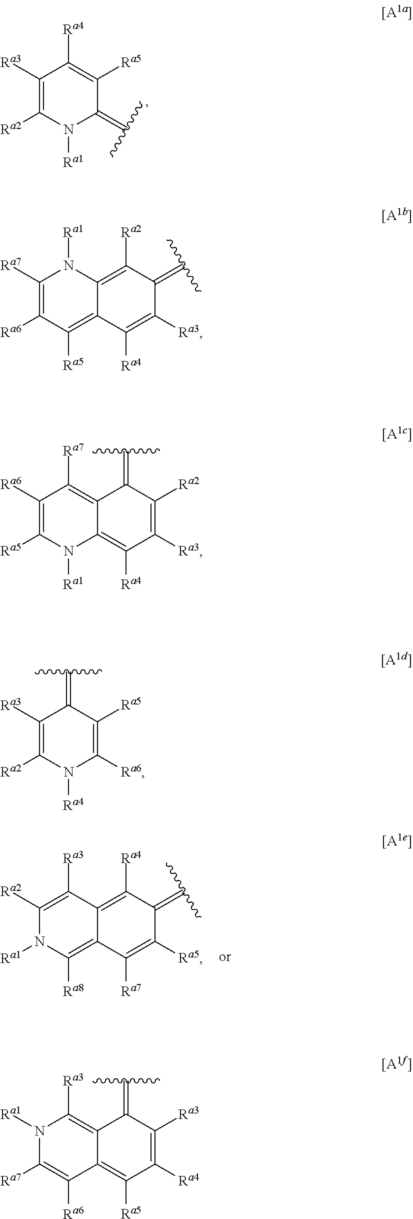

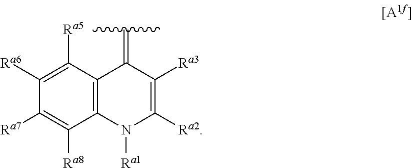

wherein R^{a1} is C₁₋₈alkyl, C₁₋₈alkylaryl, C₁₋₈alkoxy, C₁₋₈alkoxyaryl, C₂₋₈alkenyl, C₂₋₈alkynyl, aryl, C₁₋₈alkyl-C₁₋₈heteroaryl, C₃₋₈cycloalkyl, or C₁₋₈heterocyclyl;

R^{a2}, is F, Cl, Br, I, NO₂, CN, R^{a2}′, OR^{a2}′, N(R^{a2}′)₂, SO₂R^{a2}′, SO₂N(R)₂, C(O)R^{a2}′; C(O)OR^{a2}′, OC(O)R^{a2}′; C(O)N(R^{a2}′)₂, N(R^{a2}′)C(O)R^{a2}′, OC(O)N(R^{a2}′)₂, N(R^{a2}′)C(O)N(R^{a2}′)₂, wherein R^{a2}′ is in each case independently selected from hydrogen, C₁₋₈alkyl, C₁₋₈alkoxy, C₂₋₈alkenyl, C₂₋₈alkynyl, aryl, C₁₋₈heteroaryl, C₃₋₈cycloalkyl, or C₁₋₈heterocyclyl;

R^{a3} is F, Cl, Br, I, NO₂, CN, R^{a3}′, OR^{a3}′, N(R^{a3}′)₂, SO₂R^{a3}′, SO₂N(R^{a3}′)₂, C(O)R^{a3}′; C(O)OR^{a3}′, OC(O)R^{a3}′; C(O)N(R^{a3}′)₂, N(R^{a3}′)C(O)R^{a3}′, OC(O)N(R^{a3}′)₂, N(R^{a3}′)C(O)N(R^{a3}′)₂, wherein R^{a3}′ is in each case independently selected from hydrogen, C₁₋₈alkyl, C₁₋₈alkoxy, C₂₋₈alkenyl, C₂₋₈alkynyl, aryl, C₁₋₈heteroaryl, C₃₋₈cycloalkyl, or C₁₋₈heterocyclyl;

R^{a4} is F, Cl, Br, I, NO₂, CN, R^{a4}′, OR^{a4}′, N(R^{a4}′)₂, SO₂N(R^{a4}′)₂, C(O)R^{a4}′; C(O)OR^{a4}′, OC(O)R^{a4}′; C(O)N(R^{a4}′)₂, N(R^{a4}′)C(O)R^{a4}′, OC(O)N(R^{a4}′)₂, N(Rₐ₄′)C(O)N(R^{a4}′)₂, wherein R^{a4}′ is in each case independently selected from hydrogen, C₁₋₈alkyl, C₁₋₈alkoxy, C₂₋₈alkenyl, C₂₋₈alkynyl, aryl, C₁₋₈heteroaryl, C₃₋₈cycloalkyl, or C₁₋₈heterocyclyl;

R^{a5} is F, Cl, Br, I, NO₂, CN, R^{a5}′, OR^{a5}′, N(R^{a5}′)₂, SO₂R^{a5}′, SO₂N(R^{a5}′)₂, C(O)R^{a5}′; C(O)OR^{a5}′, OC(O)R^{a5}′; C(O)N(R^{a5}′)₂, N(R^{a5}′)C(O)R^{a5}′, OC(O)N(R^{a5}′)₂, N(R^{a5}′)C(O)N(R^{a5}′)₂, wherein R^{a5}′ is in each case independently selected from hydrogen, C₁₋₈alkyl, C₁₋₈alkoxy, C₂₋₈alkenyl, C₂₋₈alkynyl, aryl, C₁₋₈heteroaryl, C₃₋₈cycloalkyl, or C₁₋₈heterocyclyl;

R^{a6} is F, Cl, Br, I, NO₂, CN, R^{a6}′, OR^{a6}′, N(R^{a6}′)₂, SO₂R^{a6}′, SO₂N(R^{a6}′)₂, C(O)R^{a6}′; C(O)OR^{a6}′, OC(O)R^{a6}′; C(O)N(R^{a6}′)₂, N(R^{a6}′)C(O)R^{a6}′, OC(O)N(R^{a6}′)₂, N(R^{a6}′)C(O)N(R^{a6}′)₂, wherein R^{a6}′ is in each case independently selected from hydrogen, C₁₋₈alkyl, C₁₋₈alkoxy, C₂₋₈alkenyl, C₂₋₈alkynyl, aryl, C₁₋₈heteroaryl, C₃₋₈cycloalkyl, or C₁₋₈heterocyclyl;

R^{a7} is F, Cl, Br, I, NO₂, CN, R^{a7}′, N(R^{a7}′)₂, SO₂R^{a7}′, SO₂N(R^{a7}′)₂, C(O)R^{a7}′; C(O)OR^{a7}′, OC(O)R^{a7}′; C(O)N(R^{a7}′)₂, N(R^{a7}′)C(O)R^{a7}′, OC(O)N(R^{a7}′)₂, N(R^{a7}′)C(O)N(R^{a7}′)₂, wherein R^{a7}′ is in each case independently selected from hydrogen, C₁₋₈alkyl, C₁₋₈alkoxy, C₂₋₈alkenyl, C₂₋₈alkynyl, aryl, C₁₋₈heteroaryl, C₃₋₈cycloalkyl, or C₁₋₈heterocyclyl

R^{a8} is F, Cl, Br, I, NO₂, CN, R^{a8}′, OR^{a8}′, N(R^{a8}′)₂, SO₂N(R^{a8}′)₂, C(O)R^{a8}′; C(O)OR^{a8}′, OC(O)R^{a8}′; C(O)N(R^{a8})₂, N(R^{a8}′)C(O)R^{a8}′, OC(O)N(R^{a8}′)₂, N(R^{a8}′)C(O)N(R^{a8}′)₂, wherein R^{a8}′ is in each case independently selected from hydrogen, C₁₋₈alkyl, C₁₋₈alkoxy, C₂₋₈alkenyl, C₂₋₈alkynyl, aryl, C₁₋₈heteroaryl, C₃₋₈cycloalkyl, or C₁₋₈heterocyclyl.

In some embodiments, A² can be a heterocycle having the formula represented by A^{2a}, A^{2b}, A^{2c}, A^{2d}, A^{2e}, A^{2f} or A^{2g}:

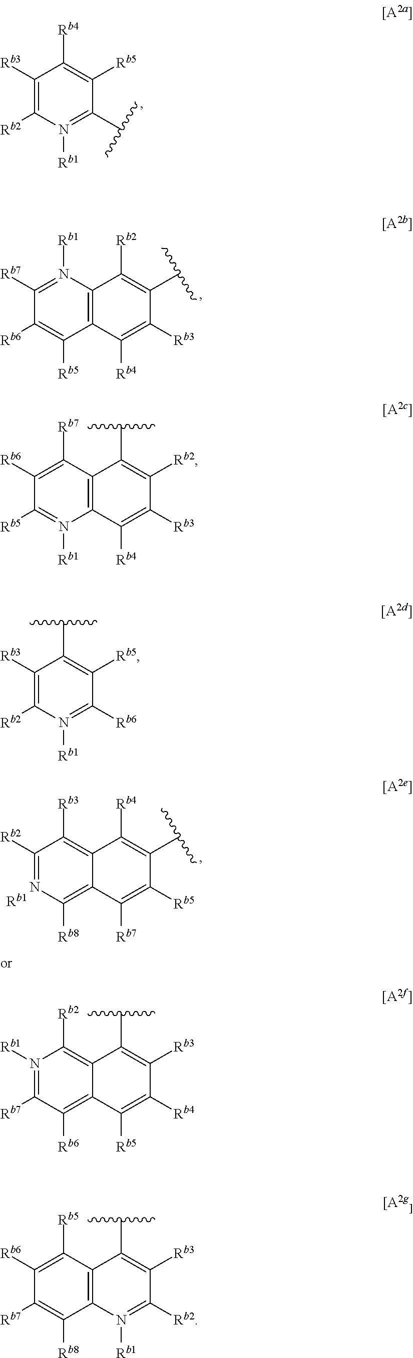

wherein R^{b1} is C₁₋₈alkyl, C₁₋₈alkylaryl, C₁₋₈alkoxy, C₁₋₈alkoxyaryl, C₂₋₈alkenyl, C₂₋₈alkynyl, aryl, C₁₋₈alkyl-C₁₋₈heteroaryl, C₃₋₈cycloalkyl, or C₁₋₈heterocyclyl;

R^{b2} is F, Cl, Br, I, NO₂, CN, R^{b2}′, OR^{b2}′, N(R^{b2}′)₂, SO₂R^{b2}′, SO₂N(R^{b2}′)₂, C(O)R^{b2}′; C(O)OR^{b2}′, OC(O)R^{b2}′; C(O)N(R^{b2}′)₂, N(R^{b2}′)C(O)R^{b2}′, OC(O)N(R^{b2}′)₂, N(R^{b2}′)C(O)N(R^{b2}′)₂, wherein R^{b2}′ is in each case independently selected from hydrogen, C₁₋₈alkyl, C₁₋₈alkoxy, C₂₋₈alkenyl, C₂₋₈alkynyl, aryl, C₁₋₈heteroaryl, C₃₋₈cycloalkyl, or C₁₋₈heterocyclyl;

R^{b3} is F, Cl, Br, I, NO₂, CN, R^{b3}′, OR^{b3}′, N(R^{b3}′)₂, SO₂R^{b3}′, SO₂N(R^{b3}′)₂, C(O)R^{b3}′; C(O)OR^{b3}′, OC(O)R^{b3}′; C(O)N(R^{b3}′)₂, N(R^{b3}′)C(O)R^{b3}′, OC(O)N(R^{b3}′)₂, N(R^{b3}′)C(O)N(R^{b3}′)₂, wherein R^{b3}′ is in each case independently selected from hydrogen, C₁₋₈alkyl, C₁₋₈alkoxy, C₂₋₈alkenyl, C₂₋₈alkynyl, aryl, C₁₋₈heteroaryl, C₃₋₈cycloalkyl, or C₁₋₈heterocyclyl;

R^{b4} is F, Cl, Br, I, NO₂, CN, R^{b4}′, N(R^{b4}′)₂, SO₂R^{b4}′, SO₂N(R^{b4}′)₂, C(O)R^{b4}′; C(O)OR^{b4}′, OC(O)R^{b4}′; C(O)N(R^{b4})₂, N(R^{b4}′)C(O)R^{b4}′, OC(O)N(R^{b4}′)₂, N(R^{b4}′)C(O)N(R^{b4}′)₂, wherein R^{b4}′ is in each case independently selected from hydrogen, C₁₋₈alkyl, C₁₋₈alkoxy, C₂₋₈alkenyl, C₂₋₈alkynyl, aryl, C₁₋₈heteroaryl, C₃₋₈cycloalkyl, or C₁₋₈heterocyclyl;

R^{b5} is F, Cl, Br, I, NO₂, CN, R^{b5}′, OR^{b5}′, N(R^{b5}′)₂, SO₂R^{b5}′, SO₂N(R^{b5}′)₂, C(O)R^{b5}′; C(O)OR^{b5}′, OC(O)R^{b5}′; C(O)N(R^{b5}′)₂, N(R^{b5}′)C(O)R^{b5}′, OC(O)N(R^{b5}′)₂, N(R^{b5}′)C(O)N(R^{b5}′)₂, wherein R^{b5}′ is in each case independently selected from hydrogen, C₁₋₈alkyl, C₁₋₈alkoxy, C₂₋₈alkenyl, C₂₋₈alkynyl, aryl, C₁₋₈heteroaryl, C₃₋₈cycloalkyl, or C₁₋₈heterocyclyl;

R^{b6} is F, Cl, Br, I, NO₂, CN, R^{b6}′, OR^{b6}′, N(R^{b6}′)₂, SO₂R^{b6}′, SO₂N(R^{b6}′)₂, C(O)R^{b6}′; C(O)OR^{b6}′, OC(O)R^{b6}′; C(O)N(R^{b6}′)₂, N(R^{b6}′)C(O)R^{b6}′, OC(O)N(R^{b6}′)₂, N(R^{b6}′)C(O)N(R^{b6}′)₂, wherein R^{b6}′ is in each case independently selected from hydrogen, C₁₋₈alkyl, C₁₋₈alkoxy, C₂₋₈alkenyl, C₂₋₈alkynyl, aryl, C₁₋₈heteroaryl, C₃₋₈cycloalkyl, or C₁₋₈heterocyclyl;

R^{b7} is F, Cl, Br, I, NO₂, CN, OR^{b7}′, N(R^{b7}′)₂, SO₂R^{b7}′, SO₂N(R^{b7}′)₂, C(O)R^{b7}′; C(O)OR^{b7}′, OC(O)R^{b7}′; C(O)N(R^{b7}′)₂, N(R^{b7}′)C(O)R^{b7}′, OC(O)N(R^{b7})₂, N(R^{b7}′)C(O)N(R^{b7}′)₂, wherein R^{b7}′ is in each case independently selected from hydrogen, C₁₋₈alkyl, C₁₋₈alkoxy, C₂₋₈alkenyl, C₂₋₈alkynyl, aryl, C₁₋₈heteroaryl, C₃₋₈cycloalkyl, or C₁₋₈heterocyclyl;

R^{b8} is F, Cl, Br, I, NO₂, CN, R^{b8}′, N(R^{b8}′)₂, SO₂R^{b8}′, SO₂N(R^{b8}′)₂, C(O)R^{b8}′; C(O)OR^{b8}′, OC(O)R^{b8}′; C(O)N(R^{b8}′)₂, N(R^{b8}′)C(O)R^{b8}′, OC(O)N(R^{b8}′)₂, N(R^{b8}′)C(O)N(R^{b8}′)₂, wherein R^{b8}′ is in each case independently selected from hydrogen, C₁₋₈alkyl, C₁₋₈alkoxy, C₂₋₈alkenyl, C₂₋₈alkynyl, aryl, C₁₋₈heteroaryl, C₃₋₈cycloalkyl, or C₁₋₈heterocyclyl.

wherein any two or more of R^{a1}, R^{a2}, R^{a3}, R^{a4}, R^{a5}, R^{a6}, R^{a7}, R^{a8}, R, R^{b1}, R^{b2}, R^{b3}, R^{b3}, R^{b4}, R^{b5}, R^{b6}, R^{b7}, and R^{b8} may together form a ring.

In certain embodiments, R^{a1} can be a C₁₋₈alkyl group substituted with mitochondria target moiety, e.g., a triphenylphosphonium salt. For instance R^{a1} can be CH₂CH₂—PPh₃, CH₂CH₂CH₂—PPh₃, CH₂CH₂CH₂CH₂—PPh₃, and the like.

In certain embodiments, when R^{a1} is C₁₋₈alkylaryl, it is preferred that it is benzyl, e.g., CH₂-phenyl. The phenyl ring may be unsubstituted, or may be substituted one or more times by groups such as F, Cl, Br, I, NO₂, CN, R^{aa}, OR^{aa}, N(R^{aa})₂, SO₂R^{aa}, SO₂N(R^{aa})₂, C(O)R^{aa}; C(O)OR^{aa}, OC(O)R^{aa}; C(O)N(R^{aa})₂, N(R^{aa})C(O)R^{aa}, OC(O)N(R^{aa})₂, N(R^{aa})C(O)N(R^{aa})₂, wherein R^{aa} is in each case independently selected from hydrogen, C₁₋₈alkyl, C₁₋₈alkoxy, C₂₋₈alkenyl, C₂₋₈alkynyl, aryl, C₁₋₈heteroaryl, C₃₋₈cycloalkyl, or C₁₋₈heterocyclyl. For instance, Rat may be a group having the formula:

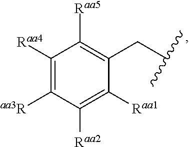

wherein:

R^{aa1} is selected from hydrogen, F, Cl, Br, I, NO₂, CN, COOH, C₁₋₈alkyl, C₁₋₈alkoxy, C₂₋₈alkenyl, C₂₋₈alkynyl, aryl, C₁₋₈heteroaryl, C₃₋₈cycloalkyl, or C₁₋₈heterocyclyl;

R^{aa2}is selected from hydrogen, F, Cl, Br, I, NO₂, CN, COOH, C₁₋₈alkyl, C₁₋₈alkoxy, C₂₋₈alkenyl, C₂₋₈alkynyl, aryl, C₁₋₈heteroaryl, C₃₋₈cycloalkyl, or C₁₋₈heterocyclyl;

R^{aa3} is selected from hydrogen, F, Cl, Br, I, NO₂, CN, COOH, C₁₋₈alkyl, C₁₋₈alkoxy, C₂₋₈alkenyl, C₂₋₈alkynyl, aryl, C₁₋₈heteroaryl, C₃₋₈cycloalkyl, or C₁₋₈heterocyclyl;

R^{aa4} is selected from hydrogen, F, Cl, Br, I, NO₂, CN, COOH, C₁₋₈alkyl, C₁₋₈alkoxy, C₂₋₈alkenyl, C₂₋₈alkynyl, aryl, C₁₋₈heteroaryl, C₃₋₈cycloalkyl, or C₁₋₈heterocyclyl;

R^{aa5} is selected from hydrogen, F, Cl, Br, I, NO₂, CN, COOH, C₁₋₈alkyl, C₁₋₈alkoxy, C₂₋₈alkenyl, C₂₋₈alkynyl, aryl, C₁₋₈heteroaryl, C₃₋₈cycloalkyl, or C₁₋₈heterocyclyl.

Preferably, one of R^{aa1}, R^{aa2}, R^{aa3}, R^{aa4}, R^{aa5} is COOH, and the remainder are hydrogen. Even more preferably, R^{aa3} is COOH and the other groups are hydrogen. In certain embodiments, when R^{a1} is C₁₋₈alkylaryl, it is preferred that it is benzyl, e.g., CH₂-phenyl. However, in other embodiments R^{a1} can be CH₂CH₂-phenyl, CH₂CH₂CH₂-phenyl, CH₂CH₂CH₂CH₂-phenyl. The phenyl ring may be unsubstituted, or may be substituted one or more times by groups such as F, Cl, Br, I, NO₂, CN, R^{aa}, OR^{aa}, N(R^{aa})₂, SO₂R^{aa}, SO₂N(R^{aa})₂, C(O)R^{aa}; C(O)OR^{aa}, OC(O)R^{aa}; C(O)N(R^{aa})₂, N(R^{aa})C(O)R^{aa}, OC(O)N(R^{aa})₂, N(R^{aa})C(O)N(R^{aa})₂, wherein R^{aa} is in each case independently selected from hydrogen, C₁₋₈alkyl, C₁₋₈alkoxy, C₂₋₈alkenyl, C₂₋₈alkynyl, aryl, C₁₋₈heteroaryl, C₃₋₈cycloalkyl, or C₁₋₈heterocyclyl.

For instance, R^{b1} may be a group having the formula:

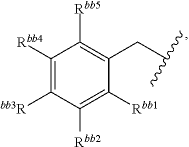

wherein:

R^{bb1} is selected from hydrogen, F, Cl, Br, I, NO₂, CN, COOH, C₁₋₈alkyl, C₁₋₈alkoxy, C₂₋₈alkenyl, C₂₋₈alkynyl, aryl, C₁₋₈heteroaryl, C₃₋₈cycloalkyl, or C₁₋₈heterocyclyl;

R^{bb2}is selected from hydrogen, F, Cl, Br, I, NO₂, CN, COOH, C₁₋₈alkyl, C₁₋₈alkoxy, C₂₋₈alkenyl, C₂₋₈alkynyl, aryl, C₁₋₈heteroaryl, C₃₋₈cycloalkyl, or C₁₋₈heterocyclyl;

R^{bb3} is selected from hydrogen, F, Cl, Br, I, NO₂, CN, COOH, C₁₋₈alkyl, C₁₋₈alkoxy, C₂₋₈alkenyl, C₂₋₈alkynyl, aryl, C₁₋₈heteroaryl, C₃₋₈cycloalkyl, or C₁₋₈heterocyclyl;

R^{bb4} is selected from hydrogen, F, Cl, Br, I, NO₂, CN, COOH, C₁₋₈alkyl, C₁₋₈alkoxy, C₂₋₈alkenyl, C₂₋₈alkynyl, aryl, C₁₋₈heteroaryl, C₃₋₈cycloalkyl, or C₁₋₈heterocyclyl;

R^{bb5} is selected from hydrogen, F, Cl, Br, I, NO₂, CN, COOH, C₁₋₈alkyl, C₁₋₈alkoxy, C₂₋₈alkenyl, C₂₋₈alkynyl, aryl, C₁₋₈heteroaryl, C₃₋₈cycloalkyl, or C₁₋₈heterocyclyl.

Preferably, one of R^{bb1}, R^{bb2}, R^{bb3}, R^{bb4}, R^{bb5} is COOH, and the remainder are hydrogen. Even more preferably, R^{bb3} is COOH and the other groups are hydrogen. In certain embodiments, when R^{a1} is C₁₋₈alkylaryl, it is preferred that it is benzyl, e.g., CH₂-phenyl. However, in other embodiments R^{b1} can be CH₂CH₂-phenyl, CH₂CH₂CH₂-phenyl, CH₂CH₂CH₂CH₂-phenyl. The phenyl ring may be unsubstituted, or may be substituted one or more times by groups such as F, Cl, Br, I, NO₂, CN, R^{bb}, OR^{bb}, N(R^{bb})₂, SO₂R^{bb}, SO₂N(R^{bb})₂, C(O)R^{bb}; C(O)OR^{bb}, OC(O)R^{bb}; C(O)N(R^{bb})₂, N(R^{bb})C(O)R^{bb}, OC(O)N(R^{bb})₂, N(R^{bb})C(O)N(R^{bb})₂, wherein R^{bb} is in each case independently selected from hydrogen, C₁₋₈alkyl, C₁₋₈alkoxy, C₂₋₈alkenyl, C₂₋₈alkynyl, aryl, C₁₋₈heteroaryl, C₃₋₈cycloalkyl, or C₁₋₈heterocyclyl.

In certain embodiments, R^{b1} can be a C₁₋₈alkyl group substituted with mitochondria target moiety, e.g., a triphenylphosphonium salt. For instance R^{b1} can be CH₂CH₂—PPh₃, CH₂CH₂CH₂—PPh₃, CH₂CH₂CH₂CH₂—PPh₃, and the like.

In certain embodiments, n is 1, i.e., a compound having the formula:

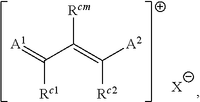

wherein A¹, A², and X have the meanings given above,

R^{c1} is selected from F, Cl, Br, I, NO₂, CN, N(R^{c1}′)₂, SO₂R^{c1}′, SO₂N(R^{c1}′)₂, C(O)R^{c1}′; C(O)OR^{c1}′, OC(O)R^{c1}′; C(O)N(R^{c1}′)₂, N(R^{c1}′)C(O)R^{c1}′, OC(O)N(R^{c1}′)₂, N(R^{c1}′)C(O)N(R^{c1}′)₂, wherein R^{c1}′ is in each case independently selected from hydrogen, C₁₋₈alkyl, C₂₋₈alkenyl, C₂₋₈alkynyl, aryl, C₁₋₈heteroaryl, C₃₋₈cycloalkyl, or C₁₋₈heterocyclyl;

R^{c2} is selected from F, Cl, Br, I, NO₂, CN, R^{c2}′, OR²′, N(R^{c2}′)₂, SO₂R^{c2}′, SO₂N(R^{c2}′)₂, C(O)R^{c2}′; C(O)OR^{c2}′, OC(O)R^{c2}′; C(O)N(R^{c2}′)₂, N(R^{c2}′)C(O)R^{c2}′, OC(O)N(R^{c2}′)₂, N(R^{c2}′)C(O)N(R^{c2}′)₂, wherein R^{c2}′ is in each case independently selected from hydrogen, C₁₋₈alkyl, C₂₋₈alkenyl, C₂₋₈alkynyl, aryl, C₁₋₈heteroaryl, C₃₋₈cycloalkyl, or C₁₋₈heterocyclyl;

R^{cm} is selected from F, Cl, Br, I, NO₂, CN, R^{cm}′, OR^{cm}′, N(R^{cm}′)₂, SO₂R^{cm}′, SO₂N(R^{cm}′)₂, C(O)R^{cm}′; C(O)OR^{cm}′, OC(O)R^{cm}′; C(O)N(R_{cm}′)₂, N(R^{cm}′)C(O)R^{cm}′, OC(O)N(R^{cm}′)₂, N(R^{cm}′)C(O)N(R^{cm}′)₂, wherein R^{cm}′ is in each case independently selected from hydrogen, C₁₋₈alkyl, C₂₋₈alkenyl, C₂₋₈alkynyl, aryl, C₁₋₈heteroaryl, C₃₋₈cycloalkyl, or C₁₋₈heterocyclyl;

wherein any two or more of A¹, R^{c1}, R^{c2}, R^{cm}, and A² may together form a ring.

Preferred R^{cm} groups include hydrogen, F, Cl, Br, optionally substituted phenyl and heteroaryl rings, and C₁₋₈alkyl-COOH groups, e.g. CH₂—COOH, CH₂CH₂—COOH, CH₂CH₂CH₂—COOH, CH₂CH₂CH₂CH₂—COOH.

In certain embodiments, n is 2, i.e., a compound having the formula:

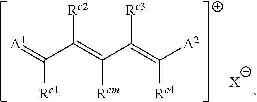

wherein A¹, A², and X have the meanings given above;

R^{c1} is selected from F, Cl, Br, I, NO₂, CN, R^{c1}′, OR^{c1}′, N(R^{c1}′)₂, SO₂R^{c1}′, SO₂N(R^{c1}′)₂, C(O)R^{c1}′; C(O)OR^{c1}′, OC(O)R^{c1}′; C(O)N(R^{c1}′)₂, N(R^{c1}′)C(O)R^{c1}′, OC(O)N(R^{c1}′)₂, N(R^{c1}′)C(O)N(R^{c1}′)₂, wherein R^{c1}′ is in each case independently selected from hydrogen, C₁₋₈alkyl, C₂₋₈alkenyl, C₂₋₈alkynyl, aryl, C₁₋₈heteroaryl, C₃₋₈cycloalkyl, or C₁₋₈heterocyclyl;

R^{c2} is selected from F, Cl, Br, I, NO₂, CN, R^{c2}′, OR^{c2}′, N(R^{c2}′)₂, SO₂R^{c2}′, SO₂N(R^{c2}′)₂, C(O)R^{c2}′; C(O)OR^{c2}′, OC(O)R^{c2}′; C(O)N(R^{c2}′)₂, N(R^{c2}′)C(O)R^{c2}′, OC(O)N(R^{c2})₂, N(R^{c2}′)C(O)N(R^{c2}′)₂, wherein R^{c2}′ is in each case independently selected from hydrogen, C₁₋₈alkyl, C₂₋₈alkenyl, C₂₋₈alkynyl, aryl, C₁₋₈heteroaryl, C₃₋₈cycloalkyl, or C₁₋₈heterocyclyl;

R^{c3} is selected from F, Cl, Br, I, NO₂, CN, R^{c3}′, OR^{c3}′, N(R^{c3}′)₂, SO₂R^{c3}′, SO₂N(R^{c3}′)₂, C(O)R^{c3}′; C(O)OR^{c3}′, OC(O)R^{c3}′; C(O)N(R^{c3}′)₂, N(R^{c3}′)C(O)R^{c3}′, OC(O)N(R^{c3}′)₂, N(R^{c3}′)C(O)N(R^{c3}′)₂, wherein R^{c3}′ is in each case independently selected from hydrogen, C₁₋₈alkyl, C₂₋₈alkenyl, C₂₋₈alkynyl, aryl, C₁₋₈heteroaryl, C₃₋₈cycloalkyl, or C₁₋₈heterocyclyl;

wherein R^{c4} is selected from F, Cl, Br, I, NO₂, CN, R^{c4}′, OR^{c4}′, N(R^{c4}′)₂, SO₂R^{c4}′, SO₂N(R^{c4}′)₂, C(O)R^{c4}′; C(O)OR^{c4}′, OC(O)R^{c4}′; C(O)N(R^{c4}′)₂, N(R^{c4}′)C(O)R^{c4}′, OC(O)N(R^{c4}′)₂, N(R^{c4}′)C(O)N(R^{c4}′)₂, wherein R^{c4}′ is in each case independently selected from hydrogen, C₁₋₈alkyl, C₂₋₈alkenyl, C₂₋₈alkynyl, aryl, C₁₋₈heteroaryl, C₃₋₈cycloalkyl, or C₁₋₈heterocyclyl;

R^{cm} is selected from F, Cl, Br, I, NO₂, CN, R^{cm}′, OR^{cm}′, N(R^{cm}′)₂, SO₂R^{cm}′, SO₂N(R^{cm}′)₂, C(O)R^{cm}′; C(O)OR^{cm}′, OC(O)R^{cm}′; C(O)N(R^{cm}′)₂, N(R^{cm}′)C(O)R^{cm}′, OC(O)N(R^{cm}′)₂, N(R^{cm}′)C(O)N(R^{cm}′)₂, wherein R^{cm}′ is in each case independently selected from hydrogen, C₁₋₈alkyl, C₂₋₈alkenyl, C₂₋₈alkynyl, aryl, C₁₋₈heteroaryl, C₃₋₈cycloalkyl, or C₁₋₈heterocyclyl.

wherein any two or more of A¹, R^{c1}, R^{c2}, R^{c3}, R^{c4}, R^{cm}, and A² may together form a ring. Preferred R^{cm} groups include hydrogen, F, Cl, Br, optionally substituted phenyl and heteroaryl rings, and C₁₋₈alkyl-COOH groups, e.g. CH₂—COOH, CH₂CH₂—COOH, CH₂CH₂CH₂—COOH, CH₂CH₂CH₂CH₂—COOH.

In certain embodiments, n is 3, i.e., a compound having the formula:

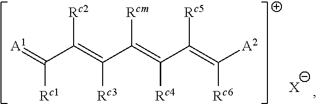

wherein A¹, A², and X have the meanings given above;

wherein R^{c1} is selected from F, Cl, Br, I, NO₂, CN, R^{c1}′, OR^{c1}′, N(R^{c1}′)₂, SO₂R^{c1}′, SO₂N(R^{c1}′)₂, C(O)R^{c1}′; C(O)OR^{c1}′, OC(O)R^{c1}′; C(O)N(R^{c1}′)₂, N(R^{c1}′)C(O)R^{c1}′, OC(O)N(R^{c1}′)₂, N(R^{c1}′)C(O)N(R^{c1}′)₂, wherein R^{c1}′ is in each case independently selected from hydrogen, C₁₋₈alkyl, C₂₋₈alkenyl, C₂₋₈alkynyl, aryl, C₁₋₈heteroaryl, C₃₋₈cycloalkyl, or C₁₋₈heterocyclyl;

R^{c2} is selected from F, Cl, Br, I, NO₂, CN, R^{c2}′, OR^{c2}′, N(R^{c2}′)₂, SO₂R^{c2}′, SO₂N(R^{c2}′)₂, C(O)R^{c2}′; C(O)OR^{c2}′, OC(O)R^{c2}′; C(O)N(R^{c2}′)₂, N(R^{c2}′)C(O)R^{c2}′, OC(O)N(R^{c2})₂, N(R^{c2}′)C(O)N(R^{c2}′)₂, wherein R^{c2}′ is in each case independently selected from hydrogen, C₁₋₈alkyl, C₂₋₈alkenyl, C₂₋₈alkynyl, aryl, C₁₋₈heteroaryl, C₃₋₈cycloalkyl, or C₁₋₈heterocyclyl;

R^{c3} is selected from F, Cl, Br, I, NO₂, CN, R^{c3}′, OR^{c3}′, N(R^{c3}′)₂, SO₂R^{c3}′, SO₂N(R^{c3}′)₂, C(O)R^{c3}′; C(O)OR^{c3}′, OC(O)R^{c3}′; C(O)N(R^{c3}′)₂, N(R^{c3}′)C(O)R^{c3}′, OC(O)N(R^{c3}′)₂, N(R^{c3}′)C(O)N(R^{c3}′)₂, wherein R^{c3}′ is in each case independently selected from hydrogen, C₁₋₈alkyl, C₂₋₈alkenyl, C₂₋₈alkynyl, aryl, C₁₋₈heteroaryl, C₃₋₈cycloalkyl, or C₁₋₈heterocyclyl;

R^{c4} is selected from F, Cl, Br, I, NO₂, CN, R^{c4}′, OR^{c4}′, N(R^{c4}′)₂, SO₂R^{c4}′, SO₂N(R^{c4}′)₂, C(O)R^{c4}′; C(O)OR^{c4}′, OC(O)R^{c4}′; C(O)N(R^{c4}′)₂, N(R^{c4}′)C(O)R^{c4}′, OC(O)N(R^{c4}′)₂, N(R^{c4}′)C(O)N(R^{c4}′)₂, wherein R^{c4}′ is in each case independently selected from hydrogen, C₁₋₈alkyl, C₂₋₈alkenyl, C₂₋₈alkynyl, aryl, C₁₋₈heteroaryl, C₃₋₈cycloalkyl, or C₁₋₈heterocyclyl;

wherein R^{c5} is selected from F, Cl, Br, I, NO₂, CN, R^{c5}′, OR^{c5}′, N(R^{c5}′)₂, SO₂R^{c5}′, SO₂N(R^{c5}′)₂, C(O)R^{c5}′; C(O)OR^{c5}′, OC(O)R^{c5}′; C(O)N(R^{c5}′)₂, N(R^{c5}′)C(O)R^{c5}′, OC(O)N(R^{c5}′)₂, N(R^{c5}′)C(O)N(R^{c5}′)₂, wherein R^{c5}′ is in each case independently selected from hydrogen, C₁₋₈alkyl, C₂₋₈alkenyl, C₂₋₈alkynyl, aryl, C₁₋₈heteroaryl, C₃₋₈cycloalkyl, or C₁₋₈heterocyclyl;

wherein R^{c6} is selected from selected from F, Cl, Br, I, NO₂, CN, R^{c6}′, OR^{c6}′, N(R^{c6}′)₂, SO₂R^{c6}′, SO₂N(R^{c6}′)₂, C(O)R^{c6}′; C(O)OR^{c6}′, OC(O)R^{c6}′; C(O)N(R^{c6}′)₂, N(R^{c6}′)C(O)R^{c6}′, OC(O)N(R^{c6}′)₂, N(R^{c6}′)C(O)N(R^{c6}′)₂, wherein R^{c6}′ is in each case independently selected from hydrogen, C₁₋₈alkyl, C₂₋₈alkenyl, C₂₋₈alkynyl, aryl, C₁₋₈heteroaryl, C₃₋₈cycloalkyl, or C₁₋₈heterocyclyl;

R^{cm} is selected from F, Cl, Br, I, NO₂, CN, R^{cm}′, OR^{cm}′, N(R^{cm}′)₂, SO₂R^{cm}′, SO₂N(R^{cm}′)₂, C(O)R^{cm}′; C(O)R^{cm}′, OC(O)R^{cm}′; C(O)N(R^{cm}′)₂, N(R^{cm}′)C(O)R^{cm}′, OC(O)N(R^{cm}′)₂, N(R^{cm}′)C(O)N(R^{cm}′)₂, wherein R^{cm}′ is in each case independently selected from hydrogen, C₁₋₈alkyl, C₂₋₈alkenyl, C₂₋₈alkynyl, aryl, C₁₋₈heteroaryl, C₃₋₈cycloalkyl, or C₁₋₈heterocyclyl, wherein any two or more of A¹, R^{c1}, R^{c2}, R^{c3}, R^{c4}, R^{c5}, R^{c6}, R^{cm}, and A²may together form a ring. Preferred R^{cm} groups include hydrogen, F, Cl, Br, optionally substituted phenyl and heteroaryl rings, and C₁₋₈alkyl-COOH groups, e.g. CH₂—COOH, CH₂CH₂—COOH, CH₂CH₂CH₂—COOH, CH₂CH₂CH₂CH₂—COOH.

In some embodiments, R^{cm} is selected from F, Cl, Br, I, aryl, C₁₋₈heteroaryl, and chelating ligands. For instance, R^{cm} can be an aryl having the formula:

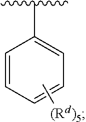

or a C₁₋₈heteroaryl having the formula:

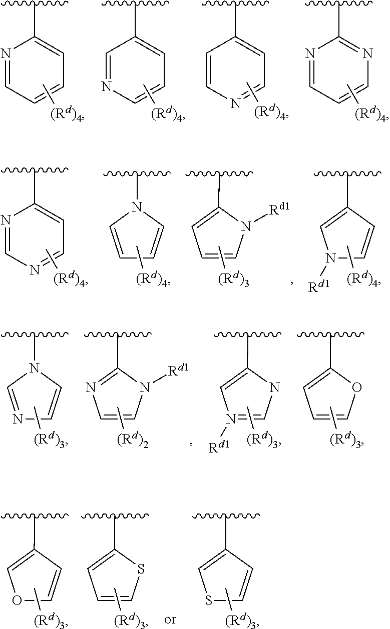

wherein R^{d} is in each case independently selected from F, Cl, Br, I, NO₂, CN, R^{d1}′, N(R^{d1}′)₂, SO₂R^{d1}′, SO₂N(R^{d1}′)₂, C(O)R^{d1}′; C(O)OR^{d1}′, OC(O)R^{d1}′; C(O)N(R^{d1}′)₂, N(R^{d1}′)C(O)R^{d1}′, OC(O)N(R^{d1}′)₂, N(R^{d1}′)C(O)N(R^{d1}′)₂, wherein R^{d1}′ is in each case independently selected from hydrogen, C₁₋₈alkyl, C₁₋₈alkoxy, C₂₋₈alkenyl, C₂₋₈alkynyl, aryl, C₁₋₈heteroaryl, C₃₋₈cycloalkyl, or C₁₋₈heterocyclyl. In a preferred embodiment, R^{d} can be a carboxylic acid, (meth)acrylate ester, azide, or aldehyde. Such groups can facilitate conjugation of the compound to a biopolymer such as a protein (including antibody), oligosaccharide, or other agent of interest.

R^{d1} is selected from hydrogen, C₁₋₈alkyl, C₂₋₈alkenyl, C₂₋₈alkynyl, aryl, C₁₋₈heteroaryl, C₃₋₈cycloalkyl, or C₁₋₈heterocyclyl; wherein any two of more of R^{d} and R^{d1} can together form a ring.

In certain case, R^{cm} is a moiety having the formula:

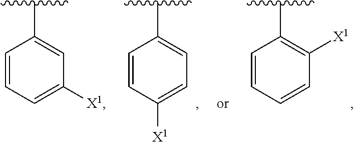

wherein X¹ is F, Cl, Br, I, NO₂, CN, R^{x1}′, OR^{x1}′, N(R^{x1}′)₂, SO₂R^{x1}′, SO₂N(R^{x1}′)₂, C(O)R^{x1}′; C(O)OR^{x1}′, OC(O)R^{x1}′; C(O)N(R^{x1}′)₂, N(R^{x1}′)C(O)R^{x1}′, OC(O)N(R^{x1}′)₂, N(R^{x1}′)C(O)N(R^{x1}′)₂, wherein R^{x1}′ is in each case independently selected from hydrogen, C₁₋₈alkyl, C₁₋₈alkoxy, C₂₋₈alkenyl, C₂₋₈alkynyl, aryl, C₁₋₈heteroaryl, C₃₋₈cycloalkyl, or C₁₋₈heterocyclyl. In some instances X¹ can include a chelating ligand as defined above. For instance, X¹ can include an ethylene glycol group of formula —(OCH₂CH₂)—OR^{L}, wherein n is an integer greater or equal to 1, for instance, 1, 2, 3, 4, 5, or 6; and R^{L} is hydrogen, C₁₋₈alkyl, C₁₋₈alkoxy, C₂₋₈alkenyl, C₂₋₈alkynyl, aryl, C₁₋₈heteroaryl, C₃₋₈cycloalkyl, or C₁₋₈heterocyclyl. Preferably, R^{L} is hydrogen or methyl. In other instances, X¹ can include a crown ether as defined above.

In some embodiments, any of R^{CM}, X¹, R^{d} and/or R^{d1} can include a chelating ligand as defined above. For instance, R^{d} and/or R^{d1} can include an ethylene glycol group of formula —(OCH₂CH₂)ₙ—OR^{L}, wherein n is an integer greater or equal to 1, for instance, 1, 2, 3, 4, 5, or 6; and R^{L} is hydrogen, C₁₋₈alkyl, C₁₋₈alkoxy, C₂₋₈alkenyl, C₂₋₈alkynyl, aryl, C₁₋₈heteroaryl, C₃₋₈cycloalkyl, or C₁₋₈heterocyclyl. Preferably, R^{L} is hydrogen or methyl. In other instances, R^{CM}, X¹, R^{d} and/or R^{d1} can include a crown ether as defined above. In some embodiments, any of R^{CM}, X¹, R^{d} and/or R^{d1} can include a mitochondria targeting ligand. For instance, a group having the formula:

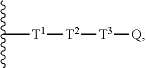

wherein T¹, T², and T³ are independently selected from null, C₁₋₈alkyl, C₁₋₈alkoxy, C₂₋₈alkenyl, C₂₋₈alkynyl, aryl, C₁₋₈heteroaryl, C₃₋₈cycloakyl, C₁₋₈heterocyclyl, an ethylene glycol group of formula —(OCH₂CH₂)ₙ— wherein n is an integer greater than 1; and Q can be:
a phosphine having the formula: —PR₃, wherein R is selected from C₁₋₈alkyl, C₁₋₈alkoxy, C₂₋₈alkenyl, C₂₋₈alkynyl, aryl, C₁₋₈heteroaryl, C₃₋₈cycloalkyl, C₁₋₈heterocyclyl, preferably R is in each case phenyl;
a dequalinium analog having the formula:

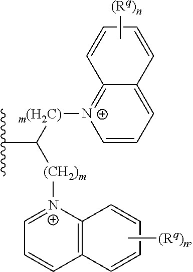

wherein m is independently selected from 5-20, n is independently selected from 0-7, and R^{q} is in each case independently selected from C₁₋₈alkyl, and NH₂,
a rhodamine having the formula:

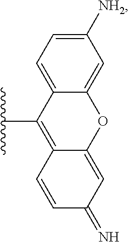 [SMILES: *c1c2ccc(=N)cc-2oc2cc(N)ccc12]

or
a tetrapeptide having the formula:

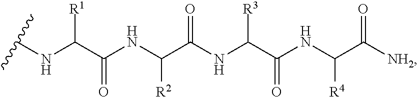

wherein R¹, R², R³ and R⁴ are independently selected from benzyl, 4-hydroxylbenzyl, 4-hydroxy-2,6-dimethylbenzyl, 3-guanidinyl-propyl, and 4-aminobutyl. In certain embodiments, R¹ and R³ are independently selected from benzyl, 4-hydroxylbenzyl, 4-hydroxy-2,6-dimethylbenzyl, and R² and R⁴ are independently selected from 3-guanidinyl-propyl, and 4-aminobutyl. In other embodiments, R² and R⁴ are independently selected from benzyl, 4-hydroxylbenzyl, 4-hydroxy-2,6-dimethylbenzyl, and R¹ and R³ are independently selected from 3-guanidinyl-propyl, and 4-aminobutyl.

In some embodiments, the compound can have the formula:

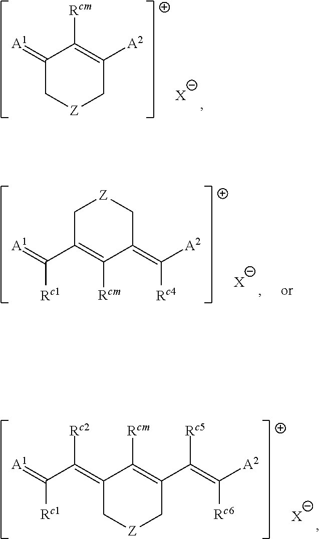

wherein A¹, R^{cm}, R^{c1}, R^{c2}, R^{cm}, R^{c5}, R^{c6}, A² and X have the aforementioned meanings, and Z is selected from null, C(R^{z4})₂, O, S, SO, SO₂, or NR^{z4}, wherein R^{z4} is independently selected from hydrogen, C₁₋₈alkyl, C₁₋₈alkoxy, C₂₋₈alkenyl, C₂₋₈alkynyl, aryl, C₁₋₈heteroaryl, C₃₋₈cycloalkyl, or C₁₋₈heterocyclyl. In some cases R^{z4} can be a chelating ligand as described above. Preferred Z groups are those that form six-membered rings, e.g., CH₂, O, NR^{z4}, S, SO, and SO₂.

Other ring systems can also be formed. For instance, R^{c1} and R^{c2} can together form a ring, e.g., an aryl ring; R^{c2} and R^{c3} can together form a ring, e.g., an aryl ring; R^{c1} and R^{cm} can together form a ring, e.g., an aryl ring; R^{c5} and R^{c6} can together form a ring, e.g., an aryl ring; R^{c4} and R^{c5} can together form a ring, e.g., an aryl ring; or R^{cm} and R^{c4} can together form a ring, e.g., an aryl ring.

In some embodiments, it is preferred that A¹ and/or A² include fused ring systems. For instance, R^{a1} and R^{a2} can together form an aryl or heteroaryl ring; R^{a2} and R^{a3} can together form an aryl or heteroaryl ring; R^{a3} and R^{a4} can together form an aryl or heteroaryl ring; R^{a4} and R^{a5} can together form an aryl or heteroaryl ring; R^{a5} and R^{a6} can together form an aryl or heteroaryl ring; R^{a6} and R^{a7} can together form an aryl or heteroaryl ring; R^{a1} and R^{a6} can together form an aryl or heteroaryl ring; or R^{a1} and R^{a7} can together form an aryl or heteroaryl ring. In certain embodiments, R^{b1} and R^{b2} can together form an aryl or heteroaryl ring; R^{b2} and R^{b3} can together form an aryl or heteroaryl ring; R^{b3} and R^{b4} can together form an aryl or heteroaryl ring; R^{b4} and R^{b5} can together form an aryl or heteroaryl ring; R^{b5} and R^{b6} can together form an aryl or heteroaryl ring; R^{b6} and R^{b7} can together form an aryl or heteroaryl ring; R^{b1} and R^{b6} can together form an aryl or heteroaryl ring; or R^{b1} and R^{b7} can together form an aryl or heteroaryl ring. The rings formed by the combination of these radicals may themselves be substituted one or more times by F, Cl, Br, I, NO₂, CN, COOH, COOC₁₋₈alkyl, C₁₋₈alkyl, C₁₋₈alkoxy, C₂₋₈alkenyl, C₂₋₈alkynyl, aryl, C₁₋₈heteroaryl, C₃₋₈cycloalkyl, or C₁₋₈heterocyclyl. For instance, A¹ can have the formula:

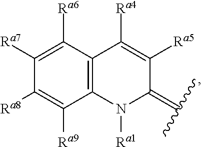

wherein R^{a8} and R^{a9} are independently selected from F, Cl, Br, I, NO₂, CN, R^{a}, OR^{a}, N(R^{a})₂, SO₂R^{a}, SO₂N(R^{a})₂, C(O)R^{a}; C(O)OR^{a}, OC(O)R^{a}; C(O)N(R^{a})₂, N(R^{a})C(O)R^{a}, OC(O)N(R^{a})₂, N(R^{a})C(O)N(R^{a})₂, wherein R^{a} is in each case independently selected from hydrogen, C₁₋₈alkyl, C₂₋₈alkenyl, C₂₋₈alkynyl, aryl, C₁₋₈heteroaryl, C₃₋₈cycloalkyl, or C₁₋₈ heterocyclyl.

In certain embodiments, it can be preferred that R^{a7} is H, F, Cl, Br, or I. In certain sub-embodiments, each of R^{a5}, R^{a6}, R^{a8}, and R^{a9} can be hydrogen, which in others, R^{a3} and R^{a4} together form a ring, e.g., an aryl ring; R^{a4} and R^{a5} together form a ring, e.g. an aryl ring; R^{a1} and R^{c1} together form a ring; R^{a5} and R^{c2} together form a ring, e.g., an aryl ring.

In certain cases, R^{a1} is a C₁₋₄alkyl group, e.g., methyl, ethyl, n-propyl, isopropyl, n-butyl, sec-butyl, isobutyl, or tert-butyl group. In other cases, R^{a1} is a benzyl group. In yet further embodiments, R^{a1} can form a ring with R^{a2}, R^{a1} and R^{a2} can together form an aryl ring; R^{a2} and R^{a1} together form a ring, e.g., an aryl ring.

In certain embodiments, A² has the formula:

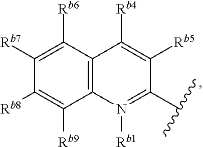

wherein R^{b8} and R^{b9} are independently selected from F, Cl, Br, I, NO₂, CN, R^{b}, OR^{b}, N(R^{b})₂, SO₂R^{b}, SO₂N(R^{b})₂, C(O)R^{b}; C(O)OR^{b}, OC(O)R^{b}; C(O)N(R^{b})₂, N(R^{b})C(O)R^{b}, OC(O)N(R^{b})₂, N(R^{b})C(O)N(R^{a})₂, wherein R^{b} is in each case independently selected from hydrogen, C₁₋₈alkyl, C₂₋₈alkenyl, C₂₋₈alkynyl, aryl, C₁₋₈heteroaryl, C₃₋₈cycloalkyl, or C₁₋₈heterocyclyl.

In certain embodiments, R^{b7} is H, F, Cl, Br, or I. In certain sub-embodiments, each of R^{b5}, R^{b6}, R^{b8}, and R^{b9} can be hydrogen, which in others, R^{b3} and R^{b4} together form a ring, e.g., an aryl ring; R^{b4} and R^{b5} together form a ring, e.g., an aryl ring; R^{b1} and R^{c5} together form a ring; R^{b5} and R^{c4} together form a ring, e.g., an aryl ring.

It can be preferred that R^{b1} is a C₁₋₄alkyl, e.g., methyl, ethyl, n-propyl, isopropyl, n-butyl, sec-butyl, isobutyl, or tert-butyl group. In some instances, R^{b1} is a benzyl group.

In some embodiments, the compounds disclosed herein may be conjugated to one or more targeting ligands to increase selectivity for target tissues. In some instances, the targeting ligand can be an antibody, for instance a monoclonal antibody, a peptide-fragment, or a small molecule. In some instances, the targeting ligand can be selective for one or more tumor or cancer types.

In some embodiments, the compounds can have the formula:

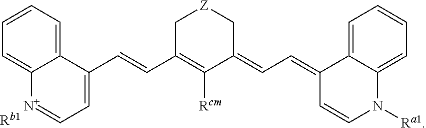

wherein R^{a1} and R^{b1} are independently selected from C₁₋₈alkyl and C₁₋₈alkylaryl, optionally substituted with one or more groups like COOH, PPh₃, OH, F, Cl, and Br; Z is selected from null, C(R^{z4})₂, O, S, SO, SO₂, or NR^{z4}, wherein R^{z4} is independently selected from hydrogen, C₁₋₈alkyl, C₁₋₈alkoxy, C₂₋₈alkenyl, C₂₋₈alkynyl, aryl, C₁₋₈heteroaryl, C₃₋₈cycloalkyl, or C₁₋₈heterocyclyl, and R^{cm} is selected from F, Cl, Br, I, C₁₋₈alkyl, aryl, C₁₋₈heteroaryl. In preferred embodiments, R^{a1} and R^{b1} are (CH₂)ₙPPh₃ (CH₂)ₙPh-4-COOH and (CH₂)ₙPh, wherein n is 1, 2, 3, 4, or 5, and Ph-4-COOH refers to a 4-yl-benzoic acid group. In preferred embodiments, Z is CH₂, and R^{cm} is selected from H, Cl, Br, aryl, heteroaryl, (CH₂)ₙPPh₃, and (CH₂)ₙCOOH, wherein n is 1, 2, 3, 4, or 5. In certain embodiments, R^{cm} can be an aryl or heteroaryl group further substituted with (CH₂)ₙPPh₃ or (CH₂)ₙCOOH.

Exemplary compounds include:

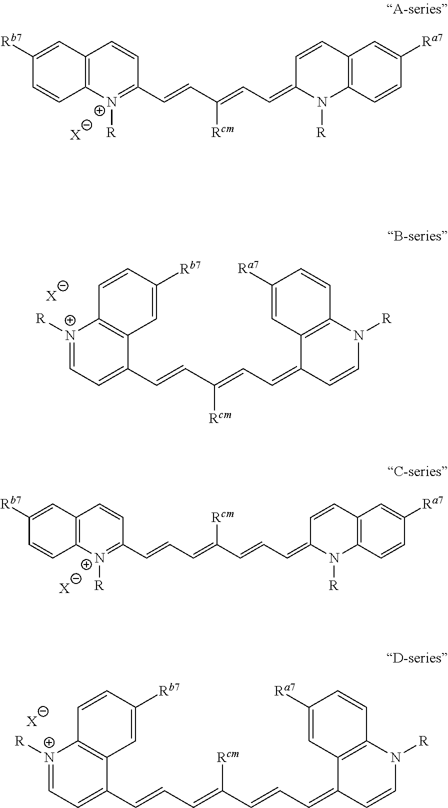

Compounds may be designated 1-A, 1-B, 1-C, 1-D, etc. . . , to reflect the substituent pattern and parent methine dye structure.

| No. | R^{cm} | R | R^{a7}; R^{b7} |
|---|---|---|---|
| 1 | H | CH₃ | H |
| 2 | Cl | CH₃ | H |
| 3 | Br | CH₃ | H |
| 4 | phenyl | CH₃ | H |
| 5 | 4-chlorophenyl | CH₃ | H |
| 6 | 4-bromophenyl | CH₃ | H |
| 7 | 4-methoxyphenyl | CH₃ | H |
| 8 | 4-fluorophenyl | CH₃ | H |
| 9 | 4-nitrophenyl | CH₃ | H |
| 10 | phenyl-4-carboxylic acid | CH₃ | H |
| 11 | 4-yl pyridine | CH₃ | H |
| 12 | 3-yl pyridine | CH₃ | H |
| 13 | 2-yl pyridine | CH₃ | H |
| 14 | CH₃ | CH₃ | H |
| 15 | H | Bn | H |
| 16 | Cl | Bn | H |
| 17 | Br | Bn | H |
| 18 | phenyl | Bn | H |
| 19 | 4-chlorophenyl | Bn | H |
| 20 | 4-bromophenyl | Bn | H |
| 21 | 4-methoxyphenyl | Bn | H |
| 22 | 4-fluorophenyl | Bn | H |
| 23 | 4-nitrophenyl | Bn | H |
| 24 | 3-iodophenyl | Bn | H |
| 25 | 4-yl pyridine | Bn | H |
| 26 | 3-yl pyridine | Bn | H |
| 27 | 2-yl pyridine | Bn | H |
| 28 | CH₃ | Bn | H |
| 29 | H | CH₃ | F |
| 30 | Cl | CH₃ | F |
| 31 | Br | CH₃ | F |
| 32 | phenyl | CH₃ | F |
| 33 | 4-chlorophenyl | CH₃ | F |
| 34 | 4-bromophenyl | CH₃ | F |
| 35 | 4-methoxyphenyl | CH₃ | F |
| 36 | 4-fluorophenyl | CH₃ | F |
| 37 | 4-nitrophenyl | CH₃ | F |
| 38 | 3-iodophenyl | CH₃ | F |
| 39 | 4-yl pyridine | CH₃ | F |
| 40 | 3-yl pyridine | CH₃ | F |
| 41 | 2-yl pyridine | CH₃ | F |
| 42 | CH₃ | CH₃ | F |
| 43 | H | Bn | F |
| 44 | Cl | Bn | F |
| 45 | Br | Bn | F |
| 46 | phenyl | Bn | F |
| 47 | 4-chlorophenyl | Bn | F |
| 48 | 4-bromophenyl | Bn | F |
| 49 | 4-methoxyphenyl | Bn | F |
| 50 | 4-fluorophenyl | Bn | F |
| 51 | 4-nitrophenyl | Bn | F |
| 52 | 3-iodophenyl | Bn | F |
| 53 | 4-yl pyridine | Bn | F |
| 54 | 3-yl pyridine | Bn | F |
| 55 | 2-yl pyridine | Bn | F |
| 56 | CH₃ | Bn | F |

In certain instances, compounds having the following structures can be used:

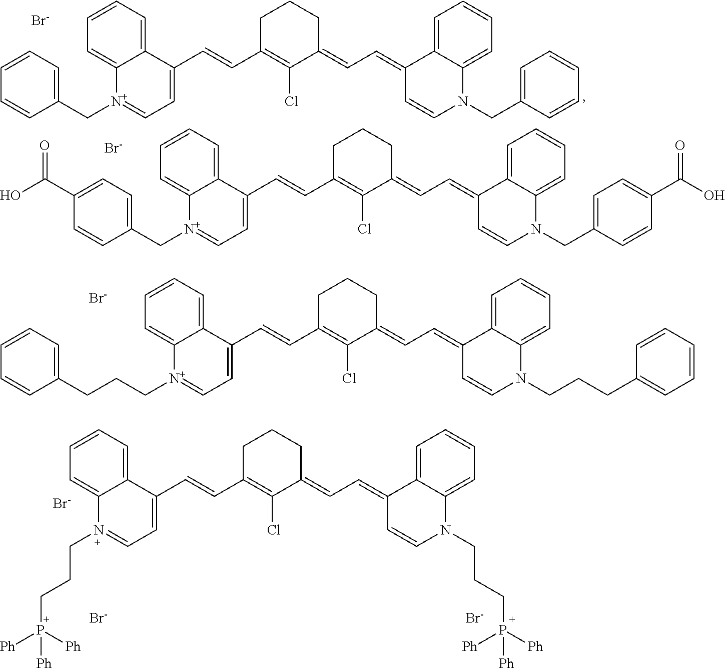

The skilled person will appreciate that while the above compounds are depicted as iodide and chloride salts, other pharmaceutically acceptable anions may also be employed.

Also disclosed are compounds having the formula:

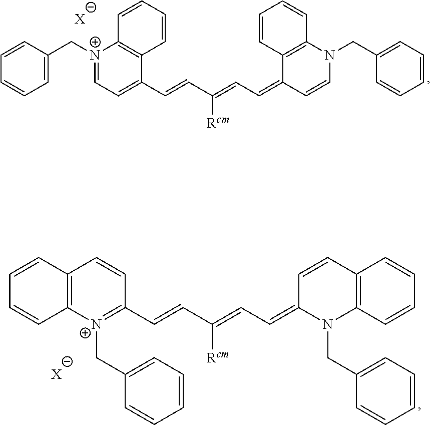

wherein R^{cm} and X are as defined above. In certain preferred embodiments, R^{cm} is H, Cl, or Br.
Efficacy in the disclosed methods include:

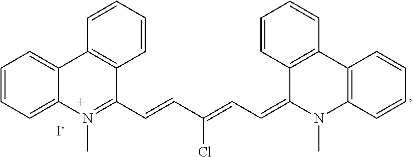

as well as the meso fluorine, bromine and iodine compound at the R^{CM} position; the compound in which R^{CM} is hydrogen has been published, however, it has not been disclosed or suggested as a compound useful for photodynamic therapy (i.e., photoinitiated cleavage of DNA);

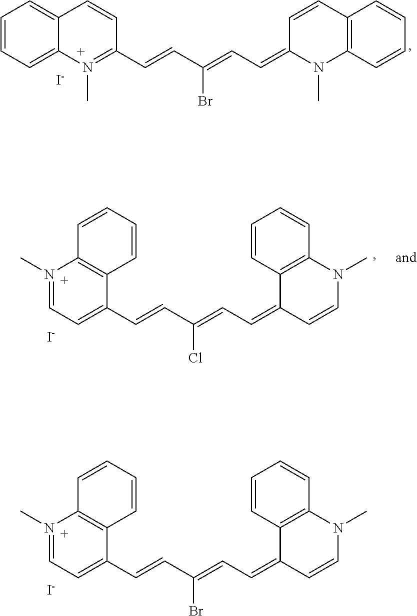

while published, these compounds have not been explored for use in photodynamic therapy (i.e., photoinitiated cleavage of DNA).

The compounds disclosed herein may be formulated in a wide variety of pharmaceutical compositions for administration to a patient. Such compositions include, but are not limited to, unit dosage forms including tablets, capsules (filled with powders, pellets, beads, mini-tablets, pills, micro-pellets, small tablet units, multiple unit pellet systems (MUPS), disintegrating tablets, dispersible tablets, granules, and microspheres, multiparticulates), sachets (filled with powders, pellets, beads, mini-tablets, pills, micro-pellets, small tablet units, MUPS, disintegrating tablets, dispersible tablets, granules, and microspheres, multiparticulates), powders for reconstitution, transdermal patches and sprinkles, however, other dosage forms such as controlled release formulations, lyophilized formulations, modified release formulations, delayed release formulations, extended release formulations, pulsatile release formulations, dual release formulations and the like. Liquid or semisolid dosage form (liquids, suspensions, solutions, dispersions, ointments, creams, emulsions, microemulsions, sprays, patches, spot-on), injection preparations, parenteral, topical, inhalations, buccal, nasal etc. may also be envisaged under the ambit of the invention.

Suitable excipients may be used for formulating the dosage forms according to the present invention such as, but not limited to, surface stabilizers or surfactants, viscosity modifying agents, polymers including extended release polymers, stabilizers, disintegrants or super disintegrants, diluents, plasticizers, binders, glidants, lubricants, sweeteners, flavoring agents, anti-caking agents, opacifiers, anti-microbial agents, antifoaming agents, emulsifiers, buffering agents, coloring agents, carriers, fillers, anti-adherents, solvents, taste-masking agents, preservatives, antioxidants, texture enhancers, channeling agents, coating agents or combinations thereof.

The compounds disclosed herein may be administered by a number of different routes. For instance, the compounds may be administered orally, topically, transdermally, intravenously, subcutaneously, by inhalation, or by intracerebroventricular delivery.

In some embodiments, the compounds disclosed herein may be formulated as nanoparticles. The nanoparticles may have an average particle size from 1-1,000 nm, preferably 10-500 nm, and even more preferably from 10-200 nm.

The compounds may be administered to a patient systemically, e.g., by oral or intravenous administration, topically, i.e., by application of a cream, lotion or the like, or locally, e.g., by direct perfusion of a composition containing the compound to a target tissue. Once administered, the compounds may be activated for DNA cleavage by targeted application of light at the relevant wavelength.

After administration of the compounds, the patient may be exposed to irradiation directed at the site of interest, e.g., a tumor. In some embodiments, the patient is irradiated with a laser having a wavelength greater than 750 nm, greater than 800 nm, greater than 850 nm, greater than 900 nm, greater than 950 nm, greater than 1000 nm, greater than 1025 nm, greater than 1050 nm, greater than 1075 nm, greater than 1100 nm, greater than 1125 nm, greater than 1150 nm, greater than 1125 nm, or greater than 1200 nm. In certain instances, because the disclosed compounds can preferentially accumulate in tumor cells and growths, a delay between administration and irradiation can be used to allow the compounds to be cleared from healthy tissues. For instance, the irradiation can take place 6 hours, 12 hours, 15 hours, 18 hours, 21 hours, 24 hours, 30 hours, 36 hours, or 48 hours after administration of the compounds.

### EXAMPLES

The following examples are for the purpose of illustration of the invention only and are not intended to limit the scope of the present invention in any manner whatsoever.

Deionized distilled water was used for buffer and DNA sample preparation. PUC19 plasmid DNA was cloned in XL-1 blue E. coli competent cells (Stratagene) according to standard laboratory protocols and was purified using a QIAfilter Plasmid Mega Kit (Qiagen™, Cat. No. 12263) by following the manufacturer's instructions. All reagents were of the highest purity available. Sonicated calf thymus (CT) DNA was obtained from Invitrogen (Cat. No. 15633-019; 10 mg/mL, average size≤2000 bp). Sodium phosphate monobasic and sodium phosphate dibasic came from Thermo Fisher Scientific. Deuterium oxide (99.9%) was supplied by Cambridge Isotope Laboratories. All other chemicals, including sodium azide (≥99.99%), sodium benzoate (99%), and dimethyl sulfoxide (DMSO, ≥99.99%) were from Sigma-Aldrich.

Synthetic Procedures

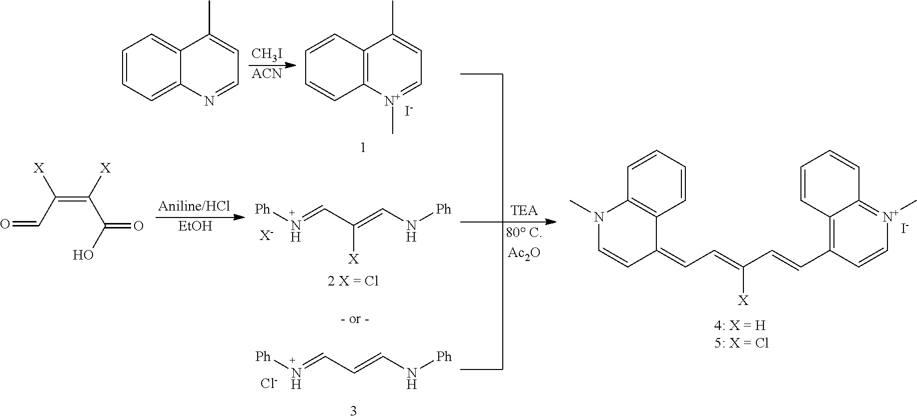

4-methylquinolinium iodide (I): Quinolinium salt 1 was obtained by the reaction of 4-methylquinoline (1 equiv) with iodomethane (4 equiv) in anhydrous acetonitrile refluxed at 90° C. for 72 h. Thin layer chromatography (TLC) was used to monitor the progress of the reaction eluting with a 4:1 mixture of DCM:hexanes. Upon completion, the reaction mixture was allowed to cool to room temperature and diethyl ether was added to precipitate the iodide salt. The solid was collected by vacuum filtration and washed with diethyl ether (3×25 mL). The salt was used without further purification in subsequent reactions.

General Procedure for the Synthesis of Polymethine Precursors

Polymethine precursor 3 was purchased from Sigma Aldrich and used as-is. Mucochloric acid (1 equiv) was dissolved in ethanol. A solution of aniline (2 equiv) was added dropwise over 10 min, and the resulting mixture was stirred and heated to 40° C. until the evolution of CO₂ (g) was observed to cease. The mixture was then cooled in an ice bath and diethyl ether was slowly added to induce precipitation of the reaction product. The resulting solid was collected by vacuum filtration, washed with diethyl ether (3×25 mL), and used without any additional purification.

Salt 1 (2 equiv) and the corresponding polymethine precursors 2 or 3 (1 equiv) were dissolved in acetic anhydride. Trimethylamine (TEA) (0.1 mL) was added and the reaction mixture was stirred and heated to 75° C. Reaction progress was monitored by UV-visible spectrophotometry. Upon completion of the reaction, diethyl ether was added to precipitate the final dyes 4 and 5, which were collected by vacuum filtration. The dyes were purified by recrystallization from methanol/diethyl ether.

1-methyl-4-((1E,3E)-5-((Z)-1 -methylquinolin-4(1H)-ylidene)penta-1,3-dien-1-yl)quinolin-1-ium iodide (4): MP 239° C. (Dec); ¹H NMR (400 MHz, DMSO-d₆) δ ppm 3.97 (s, 6 H) 6.65 (t, J=12.25 Hz, 1 H) 6.99 (d, J=13.39 Hz, 2 H) 7.32 (d, J=7.33 Hz, 2 H) 7.59 (t, J=7.07 Hz, 2 H) 7.78-7.89 (m, 4 H) 7.95 (t, J=13.01 Hz, 2 H) 8.09 (d, J=7.07 Hz, 2 H) 8.42 (d, J=8.34 Hz, 2 H); ¹³C NMR (100 MHz, DMSO-d₆) δ ppm 41.47, 107.80, 110.48, 117.22, 118.93, 124.18, 124.74, 125.90, 128.62, 132.46, 138.86, 141.36, 145.80, 146.89; HRMS (TOF MS ESI₊): calc'd for C₂₅H₂₃N₂⁺: m/z 351.1856 ([M]⁺), Found: m/z 351.0218 [M]⁺

4-((1E,3Z)-3-chloro-5-((Z)-1-methylquinolin-4(1H)-ylidene)penta-1,3-dien-1-yl)-1-methylquinolin-1-ium iodide (5): MP>260° C.; ¹H NMR (400 MHz, DMSO-d₆) δ ppm 4.05 (s, 6 H) 6.94 (d, J=13.14 Hz, 2 H) 7.41 (d, J=7.33 Hz, 2 H) 7.67 (t, J=5.81 Hz, 2 H) 7.86-7.97 (m, 4 H) 8.14 (d, J=12.88 Hz, 2 H) 8.30 (d, J=7.07 Hz, 2 H) 8.41 (d, J=8.34 Hz, 2 H); HRMS (TOF MS ESI₊): calc'd for C₂₅H₂₂ClN₂⁺: m/z 385.1466 ([M]⁺), Found: m/z 385.1964 [M]⁺.

Cyanine dyes 4 and 5 were stored in a −4° C. freezer as 2.5 mM stock solutions in DMSO.

Compound 6 (X═Br) has also been prepared.

A PerkinElmer Lambda 35 spectrophotometer, a Shimadzu UV-2401PC spectrophotometer and a PerkinElmer LS-55 fluorescence spectrometer were respectively used to record UV-visible absorption spectra and fluorescence emission spectra. Circular dichroism (CD) and induced circular dichroism (ICD) spectra were acquired with a Jasco J-810 or a Jasco J-1500 CD spectropolarimeter. At wavelengths from 190 nm to 1000 nm, the Jasco J-1500 CD spectropolarimeter was fitted with a Jasco EXPM-531 NIR extender to enhance the sensitivity of the CD signal in this range.

The absorbance of cyanine dyes was measured at 22° C. with a UV-visible spectrophotometer. Cuvettes contained 10 μM of dye in DMSO or 10 μM of dye in 10 mM of sodium phosphate pH 7.0 buffer without and with 150 μM bp CT DNA. Absorption spectra were recorded at 5 min time intervals from 0 min up to 25 or 30 min

In DNA titration experiments, small volumes of an aqueous solution of 15,111 μM bp CT DNA were sequentially added to samples containing 20 μM of cyanine dye in 10 mM sodium phosphate buffer pH 7.0 (500 μL initial volume). All absorption spectra were corrected for sample dilution. Final concentrations of CT DNA in each sample ranged from 0 μM bp up to 2684 μM bp.

Individual DNA cleavage reactions containing 5 μM to 50 μM concentrations of cyanine dye, 38 μM bp of pUC19 plasmid, and 10 mM of sodium phosphate pH 7.0 were prepared in a total volume of 40 μL. In order maintain reaction temperature at 10° C., 22° C., or 37° C., the samples were placed in a thermometer-fitted metal block that was either heated, kept at room temperature, or immersed in an ice bath. While monitoring temperature with the thermometer, the samples were either kept in the dark or were irradiated at time interval ranging from 5 min to 120 min using a light emitting diode (LED) laser (LaserLands) with a peak emission wavelength of either 532 nm (100 mW), 808 nm (300 mW), or 830 nm (300 mW). At the end of the irradiation time interval, a total of 3 μL of electrophoresis loading buffer containing 15.0% (w/v) ficoll and 0.025% (w/v) bromophenol blue) was added to each reaction and 20 μL of the resulting solution was added to one of the wells of 1.5% agarose gel stained with ethidium bromide (0.5 μg/mL, final concentration). Completely loaded gels were then electrophoresed for ˜60 min at 105 V in a Bio-Rad Laboratories gel box using 1×tris-acetate-EDTA (TAE) containing 0.5 μg/mL ethidium bromide as the running buffer. Electrophoresed gels were visualized at 302 nm with a VWR Scientific LM-20E transilluminator and then photographed with a UVP PhotoDoc-It™ Imaging System. For quantitating the gels, ImageQuant version 5.2 software was employed. The DNA photocleavage yields were then calculated using the formula:

Percent Photocleavage=[(Linear+Nicked DNA)/(Linear+Nicked+Supercoiled DNA)]×100.

### Circular Dichroism

Individual samples for CD analysis consisted of 10 mM sodium phosphate buffer pH 7.0 with 10 μM of dye and 120 μM of CT-DNA present alone and in combination (total volume of 3000 μL). Spectra were collected from 900 to 200 nm in 3 mL (1.0 cm) quartz cuvettes (Starna) using the following instrument settings: scan speed, 100 nm/min; response time, 2 s; bandwidth, 0.5 nm; sensitivity, 100 millidegrees. Final spectra were averaged over 12 acquisitions.

Extended, near-infrared circular dichroism spectra were recorded from 1000 nm to 600 nm for samples containing 10 mM sodium phosphate buffer pH 7.0, 25 μM of dye, and 990 μM bp of CT DNA (3,000 μL total volume). The scan speed was set at 100 nm/min, the response time was 2 s, and the bandwidth and sensitivity were 0.5 nm and 200 millidegrees, respectively. Final spectra were averaged over 12 acquisitions.

### Fluorescence Emission Spectra

Solutions containing 10 mM sodium phosphate buffer pH 7.0 and 10 μM of cyanine dye in the absence and presence of either 100 μM bp or 990 μM bp CT DNA were transferred to 3.0 mL Starna quartz cuvettes (3,000 μL, total volume). The samples were excited at 550 nm and 800 nm and emission spectra were respectively recorded from 555 nm to 900 nm and from 805 nm to 900 nm (22° C.).

### Reagent Induced Changes in DNA Photocleavage

In an argon-purged glove box, 40 μL photocleavage reactions containing 10 mM sodium phosphate buffer pH 7.0, 20 μM of cyanine dye, and 38 μM bp of pUC19 plasmid were prepared from solutions bubbled with argon and then irradiated at 830 nm for 30 min The procedure was repeated using aerated solutions in a glove box purged with air.

A second set of reactions containing 10 mM of sodium phosphate buffer pH 7.0, 38 μM bp pUC19 plasmid DNA and 20 μM of dye were prepared in the presence and absence of either 100 mM of the singlet oxygen scavenger sodium azide, 100 mM of the hydroxyl radical scavenger sodium benzoate, or 84% D₂O (v/v). The reactions were aerobically irradiated on the bench top for 30 min (830 nm).

After the irradiation, the above DNA reactions were electrophoresed on 1.5% non-denaturing agarose gels, visualized, and quantitated as just described. The percent change in DNA photocleavage was then calculated using the formula, where additive stands for either argon, sodium azide, sodium benzoate, or D₂O:

Percent Change in Cleavage=[(% Total of Linear and Nicked DNA_{with additive}−% Total of Linear and Nicked DNA_{without additive})/(% Total of Linear and Nicked DNA_{without additive})]×100.

DNA photocleavage inhibition induced by chemical additives

| | | Photocleavage inhibition (%) | |
|---|---|---|---|
| Reagent | Target(s) | Dye 5 | Dye 6 |
| Argon | ¹O₂ & •OH | 67 ± 3 | 75 ± 3 |
| Sodium benzoate | •OH | 31 ± 2 | 40 ± 2 |
| Sodium azide | ¹O₂ > •OH | 26 ± 3 | 24 ± 2 |
| D₂O | ¹O₂ | 6 ± 1 | 12 ± 1 |

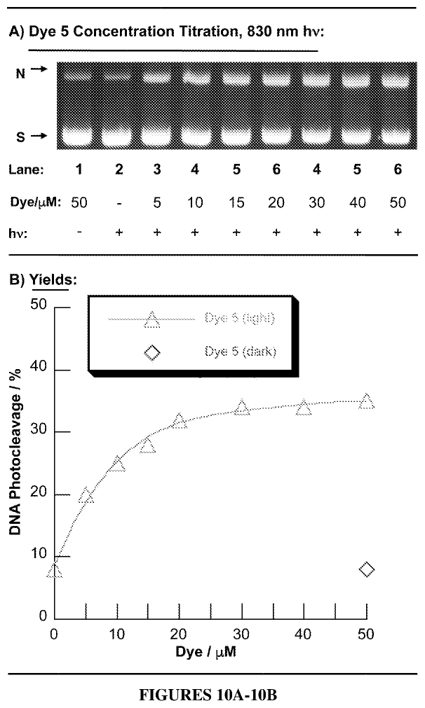

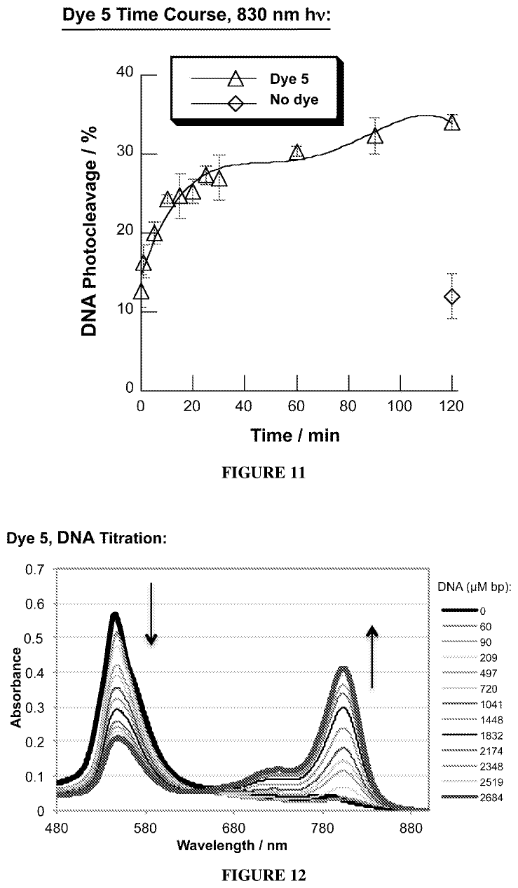

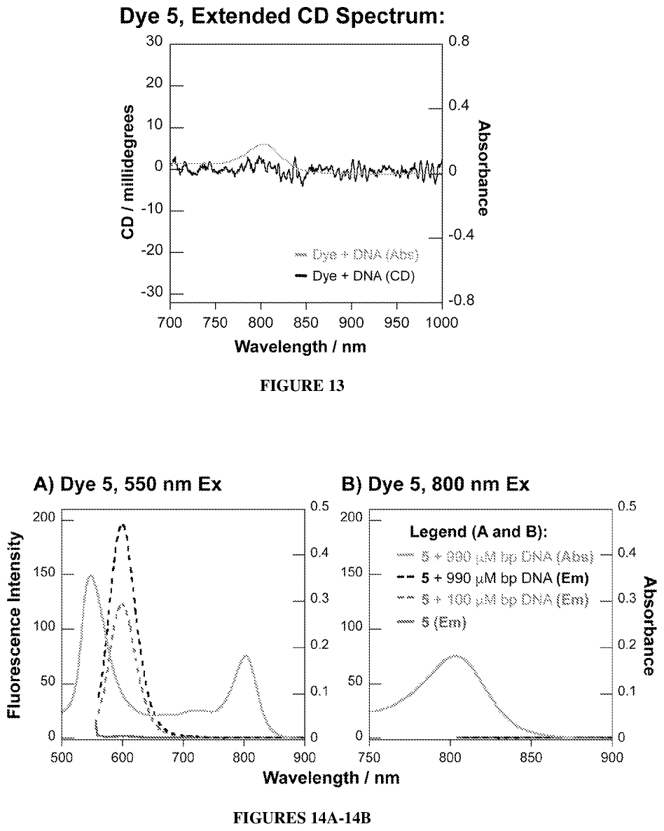

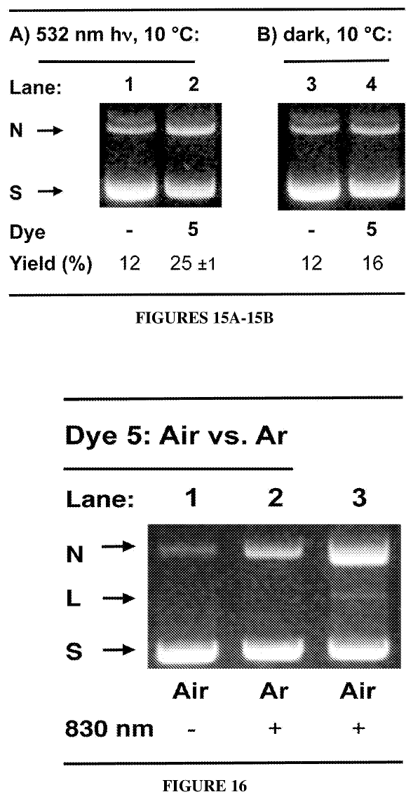

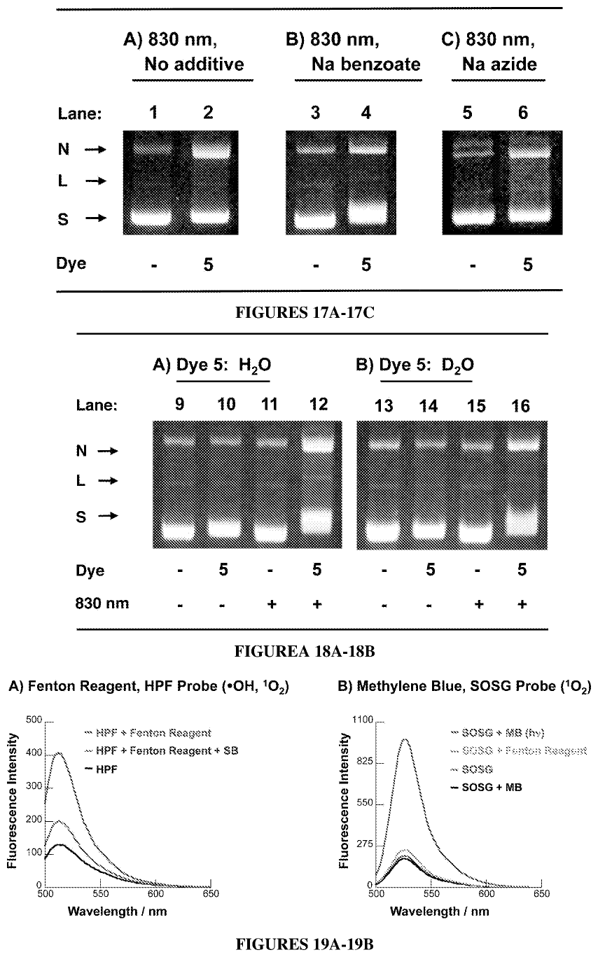

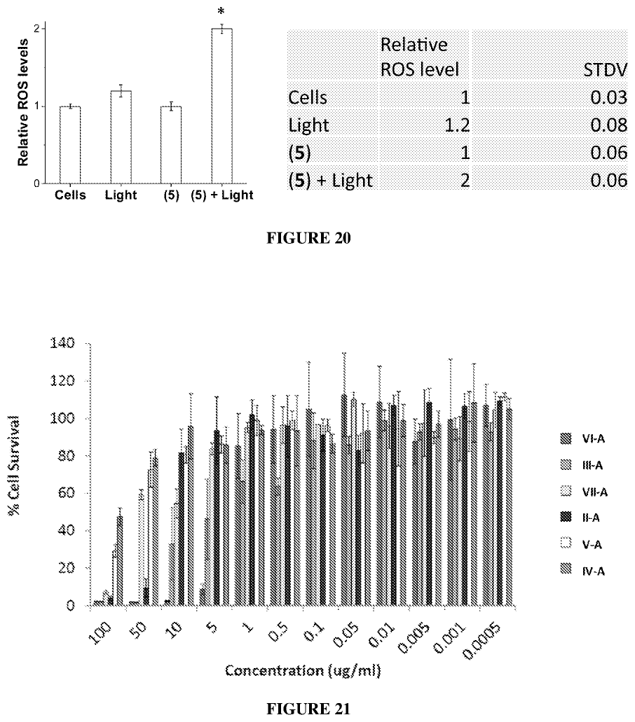

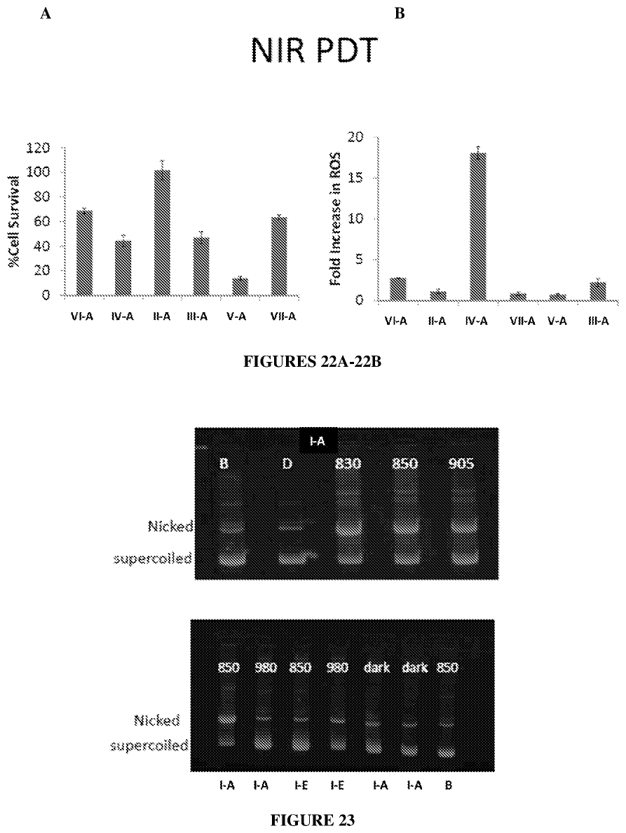

Reactions consisting of 38 μM bp of pUC19 plasmid DNA equilibrated with 20 μM of 5 or 6, with and without 100 mM of scavenger or 84% D₂O (v/v) were irradiated for 30 mM with a 830 nm, 300 mW LED laser (10 mM sodium phosphate buffer pH 7.0; FIGS. 10 through 12). Data were averaged over three trials with error reported as standard deviation.

### ROS Detection Using HPF

Solutions containing 10 mM sodium phosphate buffer pH 7.0, and 3 μM of hydroxyphenyl fluorescein (HPF) in the presence and absence of 20 μM of 5 were prepared. In a parallel reaction, a total 100 mM of sodium benzoate was used as a hydroxyl radical scavenging reagent. Samples were then kept in the dark or irradiated with an 830 nm LED laser (2.8 W/cm²) for 30 mM To generate hydroxyl radicals, aqueous solutions containing 10 μM H₂O₂, 10 μM ammonium iron(II) sulfate, 3 μM HPF, and 10 mM sodium phosphate buffer pH 7.0 in the presence and absence of 100 mM of sodium benzoate were equilibrated in the dark for a few minutes (22° C).⁵ Fluorescence emission spectra were immediately recorded using a PerkinElmer LS55 spectrofluorometer (□ₑₓ=490 nm).

### ROS Detection Using SOSG

Reactions containing 10 mM sodium phosphate buffer pH 7.0 and 0.75 μM of Singlet Oxygen Sensor Green® (SOSG) in the presence and absence of 20 μM of 5 were prepared. Samples were then kept in the dark or irradiated with an 830 nm LED laser (2.8 W/cm²) for 30 mM As a positive control for hydroxyl radical detection, an aqueous solution containing 10 μM H₂O₂, 10 μM ammonium iron(II) sulfate, 750 nM of SOSG, and 10 mM sodium phosphate buffer pH 7.0 was equilibrated in the dark for a few minutes (22° C).⁵ To generate singlet oxygen, solutions containing 1 μM of methylene blue and 10 mM sodium phosphate buffer pH 7.0 were kept in the dark or irradiated with a 638 nm LED laser (2.8 W/cm², LaserLand) for 2 s. Fluorescence emission spectra were immediately recorded with a PerkinElmer LS55 spectrofluorometer (□ₑₓ=480 nm).

### Cell Culture

ES2 human clear cell ovarian carcinoma cell line was obtained from ATCC (Manassas, Va.). All cancer cells were cultured in DMEM medium (Sigma, St. Louis, Mo.) with 10% fetal bovine serum (VWR, Visalia, Calif.) and 1.2 mL/100 mL penicillinstreptomycin (Sigma, St. Louis, Mo.). All cells were grown in a humidified atmosphere of 5% CO₂ (v/v) in air at 37° C.⁶

Cellular Uptake and Fluorescence Imaging

ES2 cells were plated in 96-well plates at a density of 10×10³ cells/well and cultured for 24 h. After that cells were incubated with the dye 5 (10 μg/mL) dissolved in DMEM (10% fetal bovine serum) for 24 h. To visualize the subcellular distribution of the dye, nuclei of ES2 cells were stained with Hoechst 33342. Before imaging, cells were washed with DPBS. Images were collected with an BZ-X710 Keyence microscope using DAPI filter and Cy® 7 filter cubes.⁷

ROS Measurements

Intracellular ROS levels were evaluated with the 2′,7′-dichlorodihydrofluorescein diacetate (DCFH-DA) assay according the previously described procedure.⁸ Briefly, ES2 cells were seeded in 96-well plates at a density of 10×10³ cells/well and cultured for 24 h. Subsequently, cells were incubated with the dye 5 dissolved in cell culture medium (1.0 μg/mL) for 24 h. Then, the cells were rinsed with DPBS and 100 μL of 10 μM DCFH-DA was added under dark conditions and incubated for 30 mM prior to light treatment. The test samples were exposed to a 808 nm laser diode light for 5 mM (0.3 W/cm²). Non-treated cells, cells incubated with the same concentration of the dye 5 under dark conditions, and cells exposed to a 808 nm laser diode for 5 mM were used as controls. Fluorescence was measured using a multiwell plate reader with 485 nm excitation and 528 nm emission filters.

Evaluation of Phototherapeutic Effect

ES2 cells were plated in 96-well plates at a density of 10×10³ cells/well and cultured for 24 h. After that cells were incubated in the dark with the dye 5 (10 μg/mL) dissolved in DMEM (10% fetal bovine serum) for 24 h. The dye-containing medium was then removed and the cells were rinsed with warm DPBS before fresh medium was added. Subsequently, cells were exposed to a 808 nm laser diode light for 10 min (0.3 W/cm²). After treatment, cells were cultured for 24 h in growth medium prior to viability measurements with Calcein AM as previously described.⁹ Non-treated cells, cells incubated with the same concentration of the dye 5 under dark conditions, and cells exposed to a 808 nm laser diode for 5 min were used as controls.

The following compounds were also evaluated:

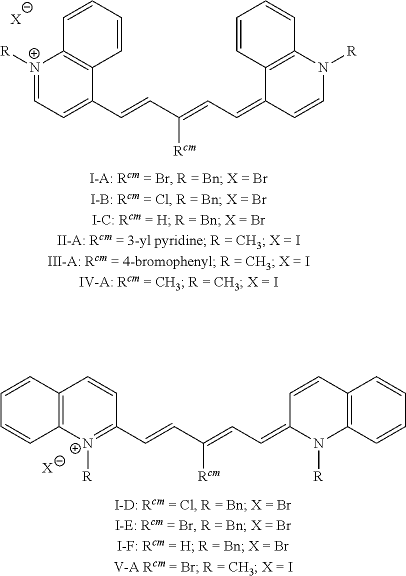

ES2 cells were plated at 10 k cells/well and left overnight. The solubilized dye was then put into the wells and left for 24 hours. The wells were then washed with DPBS and appropriate wells were exposed to the laser (5 min/well). The wells were then left in fresh media overnight. CalceinAM was then incubated was allowed to incubate for 1 hour and fluorescence was recorded on a plate reader (Synergy HT, BioTek Instruments, Winooski, Vt.) using 485 nm excitation and 528 nm emission filters.

ES2 cells were plated in a 96 well plate. 0.5 mg/ml of the dyes were placed in the wells for 24 hours.

ES2 cells were plated at 10 k cells/well and left overnight. The solubilized dye was then put into the wells and left for 24 hours. The plate was then washed with DPBS and H2DCFDA was added to each well. After 40 min, they wells were lasered and read at Read at Ex/Em: ˜492−495/517−527 nm.
IV-A 780 nm laser 0.6 W/cm2 0.90 uM
VI-A 780 nm laser 0.6 W/cm2 0.70 uM
III-A 830 nm laser 0.6 W/cm2 0.76 uM
II-A 830 nm laser 0.6 W/cm2 0.90 uM
V-A 694 nm laser 1.3 W/cm2 0.90 uM
VII-A 694 nm laser 1.3 W/cm2 0.73 uM

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein or less, however, other combinations of steps, elements, components, and constituents are included, even though not explicitly stated. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed. Other than in the examples, or where otherwise noted, all numbers expressing quantities of ingredients, reaction conditions, and so forth used in the specification and claims are to be understood at the very least, and not as an attempt to limit the application of the doctrine of equivalents to the scope of the claims, to be construed in light of the number of significant digits and ordinary rounding approaches

1-60. (canceled)

61. A compound having the formula: 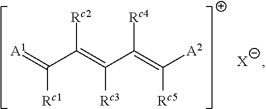
wherein X is a pharmaceutically acceptable anion.
A¹ comprises a moiety having the formula: 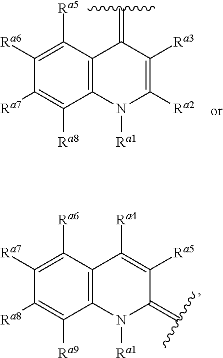
wherein
R^{a1} is C₁₋₈alkyl, C₁₋₈alkylaryl, C₁₋₈alkoxy, C₁₋₈alkoxyaryl, C₂₋₈alkenyl, C₂₋₈alkynyl, aryl, C₁₋₈alkyl-C₁₋₈heteroaryl, C₃₋₈cycloalkyl, or C₁₋₈heterocyclyl;
R^{a2}, R^{a3}, R^{a4}, R^{a5}, R^{a6}, R^{a7}, R^{a8}, and R^{a9} are independently selected from F, Cl, Br, I, NO₂, CN, R^{a}, OR^{a}, N(R^{a})₂, SO₂R^{a}, SO₂N(R^{a})₂, C(O)R^{a}; C(O)OR^{a}, OC(O)R^{a}; C(O)N(R^{a})₂, N(R^{a})C(O)R^{a}, OC(O)N(R^{a})₂, N(R^{a})C(O)N(R^{a})₂, wherein R^{a} is in each case independently selected from hydrogen, C₁₋₈alkyl, C₂₋₈alkenyl, C₂₋₈alkynyl, aryl, C₁₋₈heteroaryl, C₃₋₈cycloalkyl, or C₁₋₈heterocyclyl;
A² comprises a moiety having the formula: 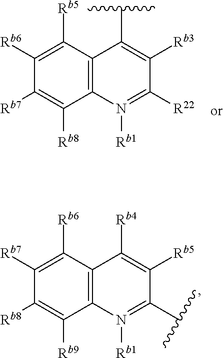
wherein
R^{b1} is C₁₋₈alkyl, C₁₋₈alkylaryl, C₁₋₈alkoxy, C₁₋₈alkoxyaryl, C₂₋₈alkenyl, C₂₋₈alkynyl, aryl, C₁₋₈alkyl-C₁₋₈heteroaryl, C₃₋₈cycloalkyl, or C₁₋₈heterocyclyl;
R^{b2}, R^{b3}, R^{b3}, R^{b4}, R^{b5}, R^{b6}, R^{b7}, R^{b8}, and R^{b9} are independently selected from F, Cl, Br, I, NO₂, CN, R^{b}, OR^{b}, N(R^{b})₂, SO₂R^{b}, SO₂N(R^{b})₂, C(O)R^{b}; C(O)OR^{b}, OC(O)R^{b}; C(O)N(R^{b})₂, N(R^{b})C(O)R^{b}, OC(O)N(R^{b})₂, N(R^{b})C(O)N(R^{b})₂, wherein R^{b} is in each case independently selected from hydrogen, C₁₋₈alkyl, C₂₋₈alkenyl, C₂₋₈alkynyl, aryl, C₁₋₈heteroaryl, C₃₋₈cycloalkyl, or C₁₋₈heterocyclyl;
R^{c1} is selected from F, Cl, Br, I, NO₂, CN, R^{c1}′, OR^{c1}′, N(R^{c1}′)₂, SO₂R^{c1}′, SO₂N(R^{c1}′)₂, C(O)R^{c1}′; C(O)OR^{c1}′, OC(O)R^{c1}′; C(O)N(R^{c1}′)₂, N(R^{c1}′)C(O)R^{c1}′, OC(O)N(R^{c1}′)₂, N(R^{c1}′)C(O)N(R^{c1}′)₂, wherein R^{c1}′ in each case independently selected from hydrogen, C₁₋₈alkyl, C₂₋₈alkenyl, C₂₋₈alkynyl, aryl, C₁₋₈heteroaryl, C₃₋₈cycloalkyl, or C₁₋₈heterocyclyl;
R^{c2} is selected from F, Cl, Br, I, NO₂, CN, R^{c2}′, OR^{c2}′, N(R^{c2}′)₂, SO₂R^{c2}′, SO₂N(R^{c2}′)₂, C(O)R^{c2}′; C(O)OR^{c2}′, OC(O)R^{c2}′; C(O)N(R^{c2}′)₂, N(R^{c2}′)C(O)R^{c2}′, OC(O)N(R^{c2}′)₂, N(R^{c2}′)C(O)N(R^{c2}′)₂, wherein R^{c2}′ is in each case independently selected from hydrogen, C₁₋₈alkyl, C₂₋₈alkenyl, C₂₋₈alkynyl, aryl, C₁₋₈heteroaryl, C₃₋₈cycloalkyl, or C₁₋₈heterocyclyl;
R^{c3} is selected from F, Cl, Br, I, NO₂, CN, R^{c3}′, OR^{c3}′, N(R^{c3}′)₂, SO₂R^{c3}′, SO₂N(R^{c3}′)₂, C(O)R^{c3}′; C(O)OR^{c3}′, OC(O)R^{c3}′; C(O)N(R^{c3}′)₂, N(R^{c3}′)C(O)R^{c3}′, OC(O)N(R^{c3}′)₂, N(R^{c3}′)C(O)N(R^{c3}′)₂, wherein R^{c3}′ is in each case independently selected from hydrogen, C₁₋₈alkyl, C₂₋₈alkenyl, C₂₋₈alkynyl, aryl, C₁₋₈heteroaryl, C₃₋₈cycloalkyl, or C₁₋₈heterocyclyl;
wherein R^{c4} is selected from F, Cl, Br, I, NO₂, CN, R^{c4}′, N(R^{c4}′)₂, SO₂R^{c4}′, SO₂N(R^{c4}′)₂, C(O)R^{c4}′; C(O)OR^{c4}′, OC(O)R^{c4}′; C(O)N(R^{c4}′)₂, N(R^{c4}′)C(O)R^{c4}′, OC(O)N(R^{c4}′)₂, N(R^{c4}′)C(O)N(R^{c4}′)₂, wherein R^{c4}′ is in each case independently selected from hydrogen, C₁₋₈alkyl, C₂₋₈alkenyl, C₂₋₈alkynyl, aryl, C₁₋₈heteroaryl, C₃₋₈cycloalkyl, or C₁₋₈heterocyclyl;
wherein R^{c5} is selected from F, Cl, Br, I, NO₂, CN, R^{c5}′, N(R^{c5}′)₂, SO₂R^{c5}′, SO₂N(R^{c5}′)₂, C(O)R^{c5}′; C(O)OR^{c5}′, OC(O)R^{c5}′; C(O)N(R^{c5}′)₂, N(R^{c5}′)C(O)R^{c5}′, OC(O)N(R^{c5}′)₂, N(R^{c5}′)C(O)N(R^{c5}′)₂, wherein R^{c5}′ is in each case independently selected from hydrogen, C₁₋₈alkyl, C₂₋₈alkenyl, C₂₋₈alkynyl, aryl, C₁₋₈heteroaryl, C₃₋₈cycloalkyl, or C₁₋₈heterocyclyl;
wherein any two or more of A¹, R^{c1}, R^{c2}, R^{c3}, R^{c4}, R^{c5}, and A² may together form a ring.

62. The compound according to claim 61, wherein R^{c1}, R^{c2}, and R^{c5} are each hydrogen, and R^{c3} is selected from F, Cl, Br, I, aryl or C₁₋₈heteroaryl.

63. The compound according to claim 61, wherein R^{c3} is selected from an aryl having the formula: 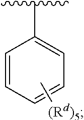
or a C₁₋₈heteroaryl having the formula: 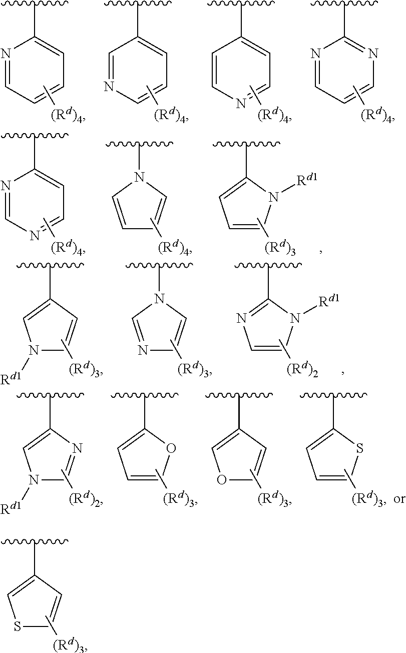
wherein R^{d} is in each case independently selected from F, Cl, Br, I, NO₂, CN, R^{d1}′, OR^{d1}′, N(R^{d1}′)₂, SO₂R^{d1}′, SO₂N(R^{d1}′)₂, C(O)R^{d1}′; C(O)OR^{d1}′, OC(O)R^{d1}′; C(O)N(R^{d1}′)₂, N(R^{d1}′)C(O)R^{d1}′, OC(O)N(R^{d1}′)₂, N(R^{d1}′)C(O)N(R^{d1}′)₂, wherein R^{d1}′ is in each case independently selected from hydrogen, C₁₋₈alkyl, C₂₋₈alkenyl, C₂₋₈alkynyl, aryl, C₁₋₈heteroaryl, C₃₋₈cycloalkyl, or C₁₋₈heterocyclyl;
R^{d1} is selected from hydrogen, C₁₋₈alkyl, C₂₋₈alkenyl, C₂₋₈alkynyl, aryl, C₁₋₈heteroaryl, C₃₋₈cycloalkyl, or C₁₋₈heterocyclyl; wherein any two of more of R^{d} and R^{d1} can together form a ring.

64. The compound of claim 63, wherein R^{c3} is a moiety having the formula: 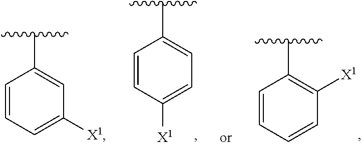
wherein X¹ is F, Cl, Br, I, NO₂, CN, R^{x1}′, OR^{x1}′, N(R^{x1}′)₂, SO₂R^{x1}′, SO₂N(R^{x1}′)₂, C(O)R^{x1}′; C(O)OR^{x1}′, OC(O)R^{x1}′; C(O)N(R^{x1}′)₂, N(R^{x1}′)C(O)R^{x1}′, OC(O)N(R^{x1}′)₂, N(R^{x1}′)C(O)N(R^{x1}′)₂, wherein R^{x1}′ is in each case independently selected from hydrogen, C₁₋₈alkyl, C₂₋₈alkenyl, C₂₋₈alkynyl, aryl, C₁₋₈heteroaryl, C₃₋₈cycloalkyl, or C₁₋₈heterocyclyl.

65. The compound according to claim 61, wherein R^{c3} is selected from F, Cl, and Br.

66. The compound according to claim 61, wherein R^{a2}, R^{a3}, R^{a4}, R^{a5}, R^{a6}, R^{a7}, R^{a8}, and R^{a9} are each hydrogen.

67. The compound according to claim 61, wherein R^{b2}, R^{b3}, R^{b4}, R^{b5}, R^{b6}, R^{b7}, R^{b8}, and R^{b9} are each hydrogen.

68. The compound according to claim 61, wherein R^{a1} and R^{b1} are each methyl.

69. A method of treating a tumor in a patient in need thereof, comprising:
(a) administering to the patient a compound according to claim 61; and
(b) irradiating the tumor with a laser having a wavelength greater than 750 nm.

70. A compound having the formula: 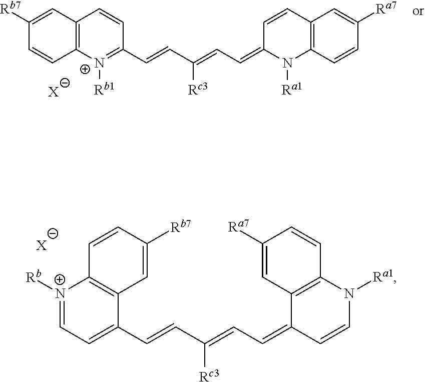
wherein
X is a pharmaceutically acceptable anion.
R^{a1} is C₁₋₈alkyl, C₁₋₈alkylaryl, C₁₋₈alkoxy, C₁₋₈alkoxyaryl, C₂₋₈alkenyl, C₂₋₈alkynyl, aryl, C₁₋₈alkyl-C₁₋₈heteroaryl, C₃₋₈cycloalkyl, or C₁₋₈heterocyclyl;
R^{b1} is C₁₋₈alkyl, C₁₋₈alkylaryl, C₁₋₈alkoxy, C₁₋₈alkoxyaryl, C₂₋₈alkenyl, C₂₋₈alkynyl, aryl, C₁₋₈alkyl-C₁₋₈heteroaryl, C₃₋₈cycloalkyl, or C₁₋₈heterocyclyl;
R^{a7} is selected from F, Cl, Br, I, NO₂, CN, R^{a}, OR^{a}, N(R^{a})₂, SO₂R^{a}, SO₂N(R^{a})₂, C(O)R^{a}; C(O)OR^{a}, OC(O)R^{a}; C(O)N(R^{a})₂, N(R^{a})C(O)R^{a}, OC(O)N(R^{a})₂, N(R^{a})C(O)N(R^{a})₂, where in R^{a} is in each case independently selected from hydrogen, C₁₋₈alkyl, C₂₋₈alkenyl, C₂₋₈alkynyl, aryl, C₁₋₈heteroaryl, C₃₋₈cycloalkyl, or C₁₋₈heterocyclyl;
R^{b7} is selected from F, Cl, Br, I, NO₂, CN, R^{b}, OR^{b}, N(R^{b})₂, SO₂R^{b}, SO₂N(R^{b})₂, C(O)R^{b}; C(O)OR^{b}, OC(O)R^{b}; C(O)N(R^{b})₂, N(R^{b})C(O)R^{b}, OC(O)N(R^{b})₂, N(R^{b})C(O)N(R^{b})₂, wherein R^{b} is in each case independently selected from hydrogen, C₁₋₈alkyl, C₂₋₈alkenyl, C₂₋₈alkynyl, aryl, C₁₋₈heteroaryl, C₃₋₈cycloalkyl, or C₁₋₈heterocyclyl;
R^{c3} is selected from Cl, Br, an aryl having the formula: 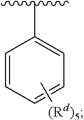
or a C₁₋₈heteroaryl having the formula: 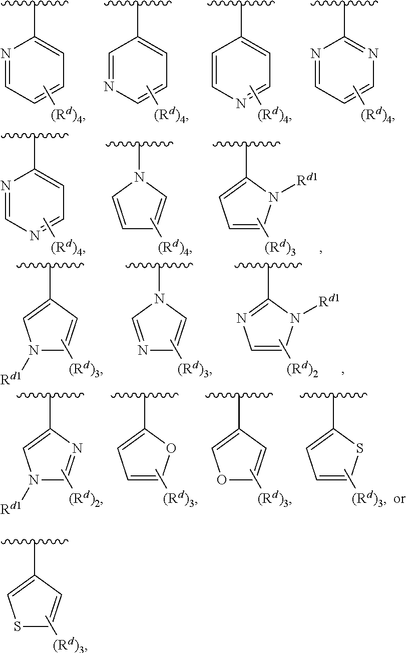
wherein R^{d} is in each case independently selected from F, Cl, Br, I, NO₂, CN, R^{d1}′, OR^{d1}′, N(R^{d1}′)₂, SO₂R^{d1}′, SO₂N(R^{d1}′)₂, C(O)R^{d1}′; C(O)OR^{d1}′, OC(O)R^{d1}′; C(O)N(R^{d1}′)₂, N(R^{d1}′)C(O)R^{d1}′, OC(O)N(R^{d1}′)₂, N(R^{d1}′)C(O)N(R^{d1}′)₂, wherein R^{d1}′ is in each case independently selected from hydrogen, C₁₋₈alkyl, C₂₋₈alkenyl, C₂₋₈alkynyl, aryl, C₁₋₈heteroaryl, C₃₋₈cycloalkyl, or C₁₋₈heterocyclyl;
R^{d1} is selected from hydrogen, C₁₋₈alkyl, C₂₋₈alkenyl, C₂₋₈alkynyl, aryl, C₁₋₈heteroaryl, C₃₋₈cycloalkyl, or C₁₋₈heterocyclyl; wherein any two of more of R^{d} and R^{d1} can together form a ring.

71. The compound according to claim 70, wherein R^{a7} and R^{b7} are each hydrogen or F.

72. The compound according to claim 70, wherein R^{a1} and R^{b1} are each C₁₋₈alkyl or C₁₋₈alkylaryl.

73. The compound according to claim 70, wherein R^{c3} is Cl or Br.

74. The compound according to claim 70, wherein R^{c3} has the formula: 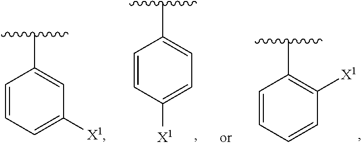
wherein X¹ is F, Cl, Br, I.

75. A compound having the formula: 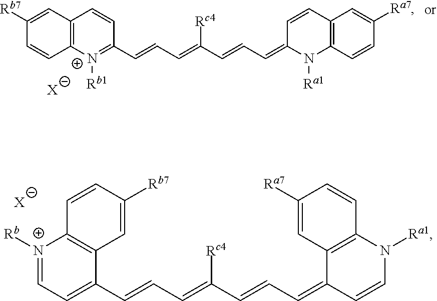
wherein
X is a pharmaceutically acceptable anion.
R^{a1} is C₁₋₈alkyl, C₁₋₈alkylaryl, C₁₋₈alkoxy, C₁₋₈alkoxyaryl, C₂₋₈alkenyl, C₂₋₈alkynyl, aryl, C₁₋₈alkyl-C₁₋₈heteroaryl, C₃₋₈cycloalkyl, or C₁₋₈heterocyclyl;
R^{b1l} is C₁₋₈alkyl, C₁₋₈alkylaryl, C₁₋₈alkoxy, C₁₋₈alkoxyaryl, C₂₋₈alkenyl, C₂₋₈alkynyl, aryl, C₁₋₈alkyl-C₁₋₈heteroaryl, C₃₋₈cycloalkyl, or C₁₋₈heterocyclyl;
R^{a7} is selected from F, Cl, Br, I, NO₂, CN, R^{a}, OR^{a}, N(R^{a})₂, SO₂R^{a}, SO₂N(R^{a})₂, C(O)R^{a}; C(O)OR^{a}, OC(O)R^{a}; C(O)N(R^{a})₂, N(R^{a})C(O)R^{a}, OC(O)N(R^{a})₂, N(R^{a})C(O)N(R^{a})₂, wherein in R^{a} is in each case independently selected from hydrogen, C₁₋₈alkyl, C₂₋₈alkenyl, C₂₋₈alkynyl, aryl, C₁₋₈heteroaryl, C₃₋₈cycloalkyl, or C₁₋₈heterocyclyl;
R^{b7} is selected from F, Cl, Br, I, NO₂, CN, R^{b}, OR^{b}, N(R^{b})₂, SO₂R^{b}, SO₂N(R^{b})₂, C(O)R^{b}; C(O)OR^{b}, OC(O)R^{b}; C(O)N(R^{b})₂, N(R^{b})C(O)R^{b}, OC(O)N(R^{b})₂, N(R^{b})C(O)N(R^{b})₂, wherein R^{b} is in each case independently selected from hydrogen, C₁₋₈alkyl, C₂₋₈alkenyl, C₂₋₈alkynyl, aryl, C₁₋₈heteroaryl, C₃₋₈cycloalkyl, or C₁₋₈heterocyclyl;
R^{c4} are selected from Cl, Br, an aryl having the formula: 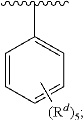
or a C₁₋₈heteroaryl having the formula: 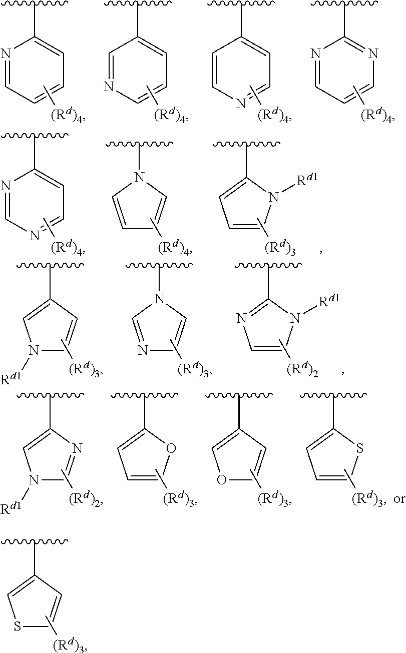
wherein R^{d} is in each case independently selected from F, Cl, Br, I, NO₂, CN, R^{d1}′, OR^{d1}′, N(R^{d1}′)₂, SO₂R^{d1}′, SO₂N(R^{d1}′)₂, C(O)R^{d1}′; C(O)OR^{d1}′, OC(O)R^{d1}′; C(O)N(R^{d1}′)₂, N(R^{d1}′)C(O)R^{d1}′, OC(O)N(R^{d1}′)₂, N(R^{d1}′)C(O)N(R^{d1}′)₂, wherein R^{d1}′ is in each case independently selected from hydrogen, C₁₋₈alkyl, C₂₋₈alkenyl, C₂₋₈alkynyl, aryl, C₁₋₈heteroaryl, C₃₋₈cycloalkyl, or C₁₋₈heterocyclyl;
R^{d1} is selected from hydrogen, C₁₋₈alkyl, C₂₋₈alkenyl, C₂₋₈alkynyl, aryl, C₁₋₈heteroaryl, C₃₋₈cycloalkyl, or C₁₋₈heterocyclyl; wherein any two of more of R^{d} and R^{d1} can together form a ring.

76. The compound according to claim 75, wherein R^{a7} and R^{b7} are each hydrogen or F.

77. The compound according to claim 75, wherein R^{a1} and R^{b1} are each C₁₋₈alkyl or C₁₋₈alkylaryl.

78. The compound according to claim 75, wherein R^{c4} is Cl or Br.

79. The compound according to claim 75, wherein R^{c4} has the formula: 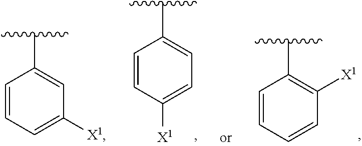
wherein X¹ is F, Cl, Br, I.

80. A method of treating a tumor in a patient in need thereof, comprising:
(a) administering to the patient a compound according to claim 75; and
(b) irradiating the tumor with a laser having a wavelength greater than 750 nm.
